(19) United States
(12) Patent Application Publication
(10) Pub. No.: US 2005/0118059 A1
(43) Pub. Date: Jun. 2, 2005

(54) Cardiopulmonary bypass extracorporeal blood circuit apparatus and method

(76) Inventors: Robert W. Olsen, Plymouth, MN (US); Walter L. Carpenter, Minneapolis, MN (US); John B. Dickey, Woods Cross, UT (US); Frederick A. Shorey JR., Grand Rapids, MI (US); Laura A. Yonce, Fridley, MN (US)

Correspondence Address: MICHAEL BEST & FRIEDRICH, LLP, 100 E WISCONSIN AVENUE, MILWAUKEE, WI, 53202, US

(21) Appl. No.: 10/978,830

(22) Filed: Nov. 1, 2004

Related U.S. Application Data
(60) Provisional application No. 60/515,619, filed on Oct. 30, 2003.

(51) Int. Cl. A61M 37/00; A61M 1/34

(52) U.S. Cl. 422/44; 604/4.01

(57) ABSTRACT

An extracorporeal blood circuit for use with a venous return line and an arterial line coupled to a patient. The extracorporeal blood circuit can include a venous air removal device coupled to the venous return line. The venous air removal device can perform an active air removal function. The extracorporeal blood circuit can include a sensor that determines a blood level in the venous air removal device, a purge line coupled to the venous air removal device, and a controller connected to the sensor. The controller can cause the venous air removal device to perform the active air removal function through the purge line when the blood level is less than a threshold. The extracorporeal blood circuit can further include a pump coupled to the venous air removal device, an oxygenator coupled to the pump, and a blood filter coupled to the oxygenator and the arterial line.

### BACKGROUND OF THE INVENTION

Conventional cardiopulmonary bypass uses an extracorporeal blood circuit that is coupled between arterial and venous cannulae and includes a venous drainage line, a venous blood reservoir, a blood pump, an oxygenator, an arterial filter, and blood transporting tubing or lines, ports, and valves interconnecting these components. Prior art, extracorporeal blood circuits as schematically depicted in FIGS. 1-3 and described in commonly assigned U.S. Pat. No. 6,302,860, draw venous blood of a patient 10 during cardiovascular surgery through the venous cannulae (not shown) coupled to venous return line 12, oxygenates the blood, and returns the oxygenated blood to the patient 10 through an arterial line 14 coupled to an arterial cannulae (not shown). Cardiotomy blood and surgical field debris that is aspirated by a suction device 16 is pumped by cardiotomy pump 18 into a cardiotomy reservoir 20.

Air can enter the extracorporeal blood circuit from a number of sources, including around the venous cannulae, through loose fittings of the lines or ports in the lines, and as a result of various unanticipated intra-operative events. It is necessary to minimize the absorption of air in the blood in the extracorporeal blood circuit and to remove any air that does accumulate in the extracorporeal blood circuit before the filtered and oxygenated blood is returned to the patient through the arterial cannulae to prevent injury to the patient. Moreover, if a centrifugal blood pump is used, a large volume of air accumulating in the venous line of the extracorporeal blood circuit can accumulate in the blood pump and either de-prime the blood pump and deprive it of its pumping capability or be pumped into the oxygenator and de-prime the oxygenator, inhibiting oxygenation of the blood.

In practice, it is necessary to initially fill the cannulae with the patient's blood and to prime (i.e., completely fill) the extracorporeal blood circuit with a biocompatible prime solution before the arterial line and the venous return lines are coupled to the blood filled cannulae inserted into the patient's arterial and venous systems, respectively. The volume of blood and/or prime solution liquid that is pumped into the extracorporeal blood circuit to “prime” it is referred to as the “prime volume.” Typically, the extracorporeal blood circuit is first flushed with CO2 prior to priming. The priming flushes out any extraneous CO2 gas from the extracorporeal blood circuit prior to the introduction of the blood. The larger the prime volume, the greater the amount of prime solution present in the extracorporeal blood circuit that mixes with the patient's blood. The mixing of the blood and prime solution causes hemodilution that is disadvantageous and undesirable because the relative concentration of red blood cells must be maintained during the operation in order to minimize adverse effects to the patient. It is therefore desirable to minimize the volume of prime solution that is required.

In one conventional extracorporeal blood circuit of the type depicted in FIG. 1, venous blood from venous return line 12, as well as de-foamed and filtered cardiotomy blood from cardiotomy reservoir 20, are discharged into a venous blood reservoir 22. Air entrapped in the venous blood rises to the surface of the blood in venous blood reservoir 22 and is vented to atmosphere through a purge line 24. The purge line 24 can be about 6 mm inner diameter flexible tubing, and the air space above the blood in venous blood reservoir 22 can be substantial. A venous blood pump 26 draws blood from the venous blood reservoir 22 and pumps it through an oxygenator 28, an arterial blood filter 30, and the arterial line 14 to return the oxygenated and filtered blood back to the patient's arterial system via the arterial cannulae coupled to the arterial line 14.

A negative pressure with respect to atmosphere is imposed upon the mixed venous and cardiotomy blood in the venous blood reservoir 22 as it is drawn by the venous blood pump 26 from the venous blood reservoir 22. The negative pressure causes the blood to be prone to entrain air bubbles. Although arterial blood filters, e.g., arterial blood filter 30, are designed to capture and remove air bubbles, they are not designed to handle larger volumes of air that may accumulate in the extracorporeal blood circuit. The arterial blood filter 30 is basically a bubble trap that traps any air bubbles larger than about 20-40 microns and discharges the air to atmosphere through a typically about 1.5 mm ID purge line 32. The arterial filter 30 is designed to operate at positive blood pressure provided by the venous blood pump 26. The arterial blood filter 30 cannot prevent accumulation of air in the venous blood pump 26 and the oxygenator 28 because it is located in the extracorporeal blood circuit downstream from them.

As shown in FIG. 2, it has been proposed to substitute an assisted venous return (AVR) extracorporeal blood circuit for the conventional extracorporeal blood circuit of the type depicted in FIG. 1, whereby venous blood is drawn under negative pressure from the patient's body. The venous blood reservoir 22, which accounts for a major portion of the prime volume of the extracorporeal blood circuit, is thereby eliminated. Furthermore, the arterial blood filter 30 is moved into the venous return line 12 upstream of the venous blood pump 26 to function as a venous blood filter. De-foamed and filtered cardiotomy blood from cardiotomy reservoir 20 is drained into the arterial blood filter 30, and venous blood in venous return line 12 and the venous cannulae coupled to it is pumped through the arterial blood filter 30. Exposure of the venous blood to air is reduced because the arterial blood filter 30 does not have an air space between its inlet and outlet (except to the extent that air accumulates above the venous blood inlet), as the venous blood reservoir 22 does. Suction is provided in the venous return line 12 through the negative pressure applied at the outlet of arterial blood filter 30 by the venous blood pump 26 to pump the filtered venous blood through the oxygenator 28 and into the arterial blood line 14 to deliver it back to patient 10. Again, the arterial blood filter 30 is basically a bubble trap that traps any air bubbles larger than about 20-40 microns and discharges the air to atmosphere through a typically about 1.5 mm inner diameter purge line 32.

The arterial blood filter 30 is relocated with respect to the cardiotomy reservoir 20 and modified to function as a venous blood filter in the extracorporeal blood circuits shown in FIGS. 3 and 4, referred to as an “AVR” extracorporeal blood circuit in the above-referenced '860 patent. Evacuation of air from venous blood received through venous return line 12 is facilitated by increasing the size of the purge port 34 of the arterial blood filter 30 to accept a larger diameter purge line 42, e.g. a 6 mm ID line, rather than the 1.5 mm ID line. A vacuum greater than that normally used for venous drainage is applied through purge line 42 to the purge port 34 to actively purge air from arterial blood filter 30. The cardiotomy reservoir 20 is at ambient pressure but is conveniently purged by the same vacuum that purges air from arterial blood filter 30. A valve 36, e.g., a one-way check valve, is incorporated into the purge port 34 or purge line 42 to prevent air or blood purged from the cardiotomy reservoir 20 from being drawn into arterial blood filter 30 by the negative pressure in arterial blood filter 30 when the purging vacuum is not active.

As shown in FIG. 4 from the above-referenced '860 patent, venous blood is drawn through the upper venous blood inlet 44, down through the filter 46 and a screen or other conventional bubble trapping device (not shown), and out the venous blood outlet 48 by the venous blood pump 26. The purge port 34 can be located above the venous blood inlet 44, and air that is separated out by the screen or other conventional bubble trapping device can accumulate in the space 50 above the venous blood inlet 44. An air sensor 38 is disposed adjacent the purge port 34 that generates a sensor signal or modifies a signal parameter in the presence of air in the space 50. The sensor signal is processed by circuitry in a controller (not shown) that applies the vacuum to the purge line 42 to draw the accumulated air out of the space 50. The vacuum is discontinued when the sensor signal indicates that venous blood is in the space 50. Thus, an “Active Air Removal” (AAR) system is provided to draw the accumulated air out of space 50 when, and only when, air present in the space 50 is detected by air sensor 34 to purge the air and to prevent venous blood filling space 50 from being aspirated out the purge line 42 by the purging vacuum. The purging vacuum may be produced by a pump 40, or it may be produced by connecting the purge line 42 to the vacuum outlet conventionally provided in operating rooms.

Again, suction is provided in the venous return line 12 through the negative pressure applied at the outlet 48 of arterial blood filter 30 by the venous blood pump 26 to pump the filtered venous blood through the oxygenator 28 and into the arterial blood line 14 to deliver it back to patient 10. De-foamed and filtered cardiotomy blood is also pumped by venous blood pump 26 from cardiotomy reservoir 20 through the oxygenator 28 and into the arterial blood line 14 to deliver it back to patient 10.

### SUMMARY OF THE INVENTION

While the AVR extracorporeal blood circuit illustrated in FIGS. 3 and 4, and particularly the use of the AAR method and system, represents a significant improvement in extracorporeal circuits, its implementation can be further refined and improved. A need remains for an AAR system and method that optimizes the air sensor and its functions and that detects and responds to error conditions and faults that can arise over the course of prolonged surgical use.

Moreover, the typical prior art extracorporeal blood circuit, e.g. the above-described extracorporeal blood circuits of FIGS. 1-3, has to be assembled in the operating room from the above-described components, primed, and monitored during the surgical procedure while the patient is on bypass. This set-up of the components can be time-consuming and cumbersome and can result in missteps that have to be corrected. Therefore, a need remains for an extracorporeal blood circuit having standardized components and that can be set up for use using standardized setup procedures minimizing the risk of error.

The resulting distribution of the components and lines about the operating table can take up considerable space and get in the way during the procedure. The connections that have to be made can also introduce air leaks introducing air into the extracorporeal blood circuit. A need remains for a compact extracorporeal blood circuit that is optimally positioned in relation to the patient and involves making a minimal number of connections.

The lengths of the interconnected lines are not optimized to minimize prime volume and attendant hemodilution and to minimize the blood contacting surface area. A large blood contacting surface area increases the incidences of embolization of blood cells and plasma traversing the extracorporeal blood circuit and complications associated with immune response, e.g., as platelet depletion, complement activation, and leukocyte activation. Therefore, a need remains for a compact extracorporeal blood circuit having minimal line lengths and minimal blood contacting surface area.

Furthermore, a need remains for such a compact extracorporeal blood circuit with minimal blood-air interfaces causing air to be entrained in the blood. In addition, it is desirable that the components be arranged to take advantage of the kinetic assisted, venous drainage that is provided by the centrifugal venous blood pump in an AVR extracorporeal blood circuit employing an AAR system.

Occasionally, it becomes necessary to “change out” one or more of the components of the extracorporeal blood circuit during the procedure. For example, it may be necessary to replace a blood pump or oxygenator. It may be necessary to prime and flush the newly constituted extracorporeal blood circuit after replacement of the malfunctioning component. The arrangement of lines and connectors may make this very difficult to accomplish. A need therefore remains for a compact extracorporeal blood circuit that can be rapidly and easily substituted for a malfunctioning extracorporeal blood circuit and that can be rapidly primed.

Consequently, a need remains for a extracorporeal blood circuit that is compactly arranged in the operating room, that takes advantage of kinetic assist, and is small in volume to minimize the required prime volume and to minimize the blood contacting surface area and blood-air interfaces. Moreover, a need remains for such an extracorporeal blood circuit that is simple to assemble and prime, provides for automatic monitoring of blood flow and other operating parameters, and facilitates change-out of components during the procedure.

One embodiment of the invention provides an extracorporeal blood circuit for use with a venous return line and an arterial line coupled to a patient. The extracorporeal blood circuit can include a venous air removal device coupled to the venous return line. The venous air removal device can perform an active air removal function. The extracorporeal blood circuit can include a sensor that determines a blood level in the venous air removal device and a purge line coupled to the venous air removal device. The extracorporeal blood circuit can include a controller connected to the sensor. The controller can cause the venous air removal device to perform the active air removal function through the purge line when the blood level is less than a threshold. The extracorporeal blood circuit can further include a pump coupled to the venous air removal device, an oxygenator coupled to the pump, and a blood filter coupled to the oxygenator and the arterial line.

Some embodiments of the invention can provide a disposable circuit support module for use with an extracorporeal blood circuit including a venous air return device, a pump, an oxygenator, and a blood filter. The disposable circuit support module can include a C-shaped arm and a plurality of snap fittings coupled to the C-shaped arm. Each one of the plurality of snap fittings can include a concave band rigidly coupled to the C-shaped arm and a movable U-shaped band that snaps into engagement with the concave band in order to engage one of the venous air return device, the oxygenator, and the blood filter.

One embodiment of the invention includes a method of priming an extracorporeal blood circuit. The method can include connecting a venous return line to an arterial line using a pre-bypass loop, preventing flow of prime solution into a venous air return device and a blood filter, and filling a pump and an oxygenator with prime solution in order to drive air bubbles upward and out of the pump and the oxygenator. The method can also include allowing prime solution to fill the venous return line and to pass into the venous return line after the pump and the oxygenator are filled with prime solution, allowing prime solution to rise upward through the venous return line into the blood filter, and coupling a vacuum source to a purge line coupled to the venous air removal device.

Embodiments of the invention provide a method of sensing and removing air and blood froth from an extracorporeal blood circuit including a venous air removal device, a pump, an oxygenator, and a blood filter. The method can include connecting at least one piezoelectric crystal to the venous air removal device and to an active air removal controller, sensing a level of blood in the venous air removal device, and controlling the venous air removal device based on the level of blood in the venous air removal device in order to automatically remove air and blood froth when the level of blood falls below a threshold level.

Other features and aspects of the invention will become apparent to those skilled in the art upon review of the following detailed description, claims and drawings.

### BRIEF DESCRIPTION OF THE DRAWINGS

FIG. 1 is a schematic diagram of a first prior art extracorporeal blood circuit that uses a venous reservoir.

FIG. 2 is a schematic diagram of a second prior art extracorporeal blood circuit that does not use a venous reservoir.

FIG. 3 is a schematic diagram of a third prior art extracorporeal blood circuit that does not use a venous reservoir and employs a venous blood filter with active air removal.

FIG. 4 is a simplified schematic view of the prior art venous blood filter of FIG. 3.

FIG. 5 is a schematic view of an extracorporeal blood circuit according to one embodiment of invention in relation to prime solution holding bags and a sequestering bag.

FIG. 6 is a perspective view of the extracorporeal blood circuit of FIG. 5 supported by a disposable circuit support module and a reusable system holder.

FIG. 7 is a perspective view of the disposable circuit support module of FIG. 6.

FIG. 8 is a schematic view of the extracorporeal blood circuit of FIG. 5 supported by the disposable circuit support module of FIGS. 6 and 7.

FIGS. 9-11 are schematic views of the extracorporeal blood circuit of FIG. 5 in relation to a sequestering bag and first and second prime solution bags and illustrating the steps of priming the disposable, integrated, extracorporeal blood circuit with prime solution.

FIGS. 12A and 12B are cross-section views of one embodiment of a Venous Air Removal Device (VARD) for use in the extracorporeal blood circuit of FIG. 5.

FIG. 13 is a schematic view of sensor elements for use in the VARD of FIGS. 12A and 12B.

FIG. 14 is a plan view of an Active Air Removal (AAR) controller for use with the extracorporeal blood circuit of FIG. 5.

FIG. 15 is a system block diagram of the AAR controller of FIG. 14.

FIGS. 16-19 are screen displays for automatic operating mode states for use with the AAR controller of FIG. 14.

FIGS. 19-46 are screen displays for automatic troubleshooting modes of operation for use with the AAR controller of FIG. 14.

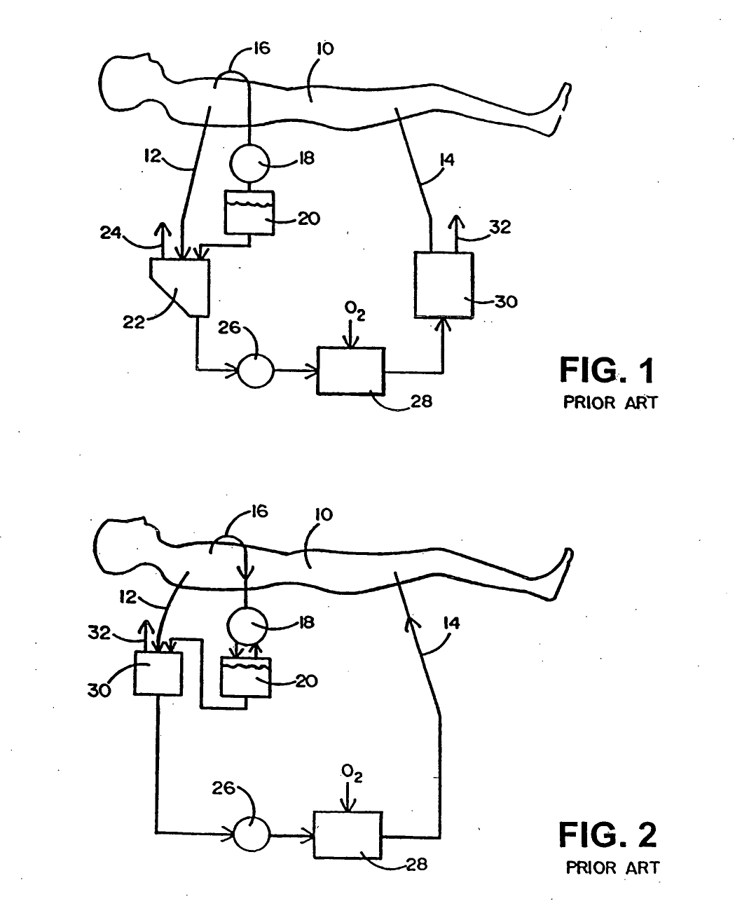

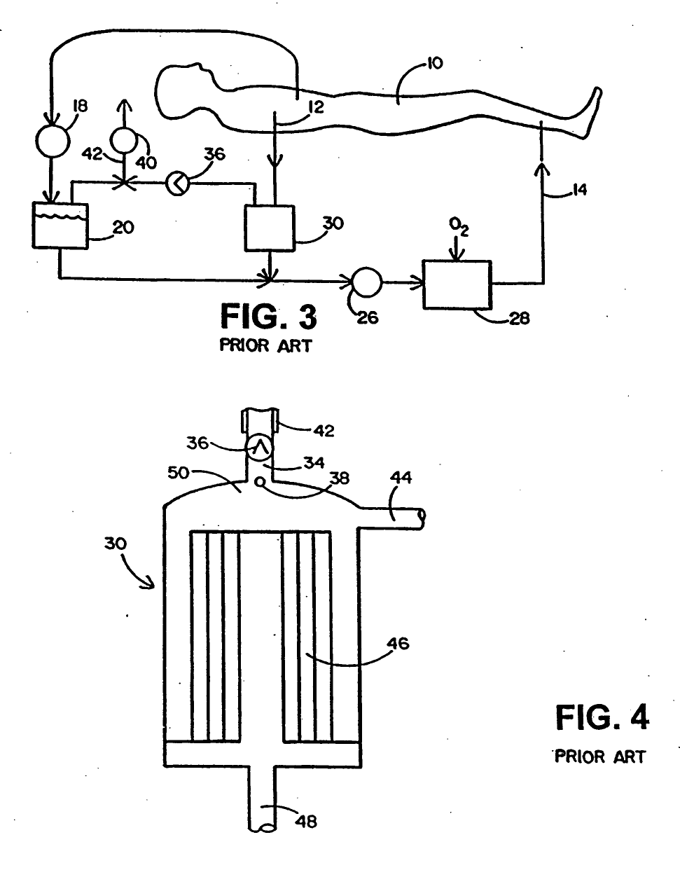

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical connections or couplings, whether direct or indirect.

In addition, it should be understood that embodiments of the invention include both hardware and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software. As such, it should be noted that a plurality of hardware and software based devices, as well as a plurality of different structural components may be utilized to implement the invention. Furthermore, and as described in subsequent paragraphs, the specific mechanical configurations illustrated in the drawings are intended to exemplify embodiments of the invention and that other alternative mechanical configurations are possible.

Some embodiments of the invention can include a method and system that incorporates a disposable, integrated, extracorporeal blood circuit with reusable components including the reusable components of a heart-lung machine. The extracorporeal blood circuit can include a Venous Air Removal Device (VARD), a centrifugal blood pump, an oxygenator, and an arterial blood filter all interconnected with fluid lines. The disposable centrifugal blood pump can be coupled with the reusable blood pump driver that can in turn be coupled to a pump driver console. Oxygen and water lines can be coupled to the disposable blood oxygenator and an oxygenator control console for controlling oxygen and water flow and water temperature. One embodiment of the VARD can include a venous filter that provides an Active Air Removal (AAR) function under the control of a reusable AAR controller. The extracorporeal blood circuit of one embodiment of the invention can include a disposable circuit support module for supporting the components and lines in a predetermined three-dimensional spatial relationship. One embodiment of the invention further comprises a reusable system holder adapted to be coupled to the reusable components of the heart lung machine to support the AAR controller and the disposable circuit support module. It will be understood that the various aspects of the invention can be practiced in alternative contexts than the context provided by the described embodiments.

The extracorporeal blood circuit of one embodiment of the invention can include access ports through which the operator or perfusionist may administer medications, fluids, and blood. In addition, the extracorporeal blood circuit can include multiple sites for sampling blood and for monitoring various parameters, e.g., temperature, pressure, and blood gas saturation. Clamps and valves can be disposed in the lines extending between or from the components of the extracorporeal blood circuit. The extracorporeal blood circuit can be set up and changed out more rapidly than conventional extracorporeal blood circuits, and arrangement of the supplied components can minimize the possibility of erroneous setup. The extracorporeal blood circuit can be a closed system that reduces the air-blood interface and that minimizes the blood contacting surface area. The extracorporeal blood circuit may be rapidly primed with prime solution. In some embodiments, the prime solution can be displaced retrograde with the patient's own blood at least in part to reduce hemodilution by the prime volume.

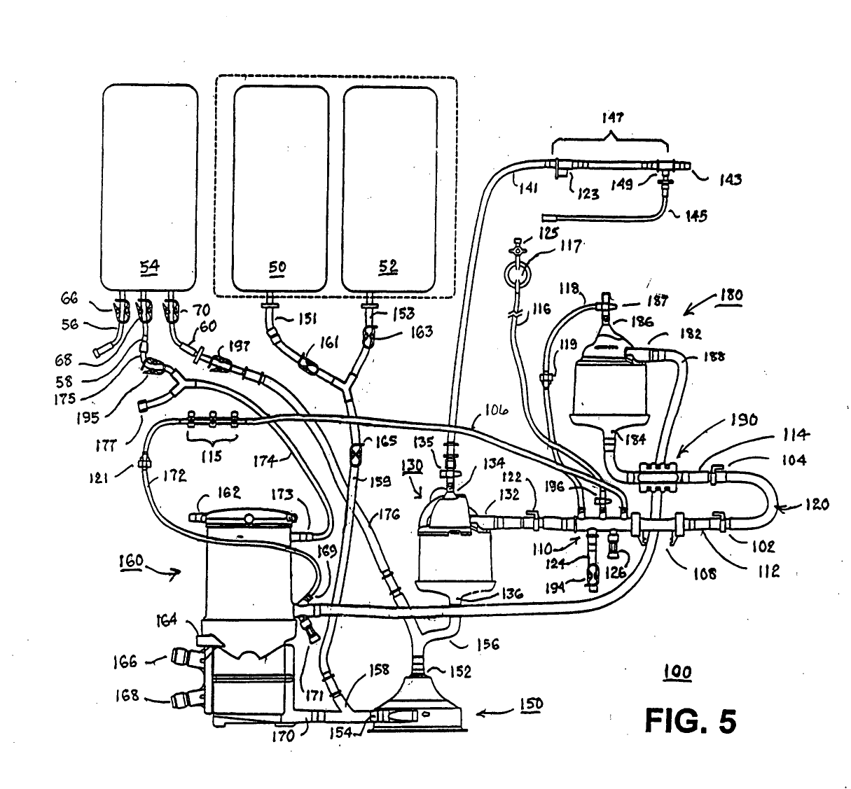

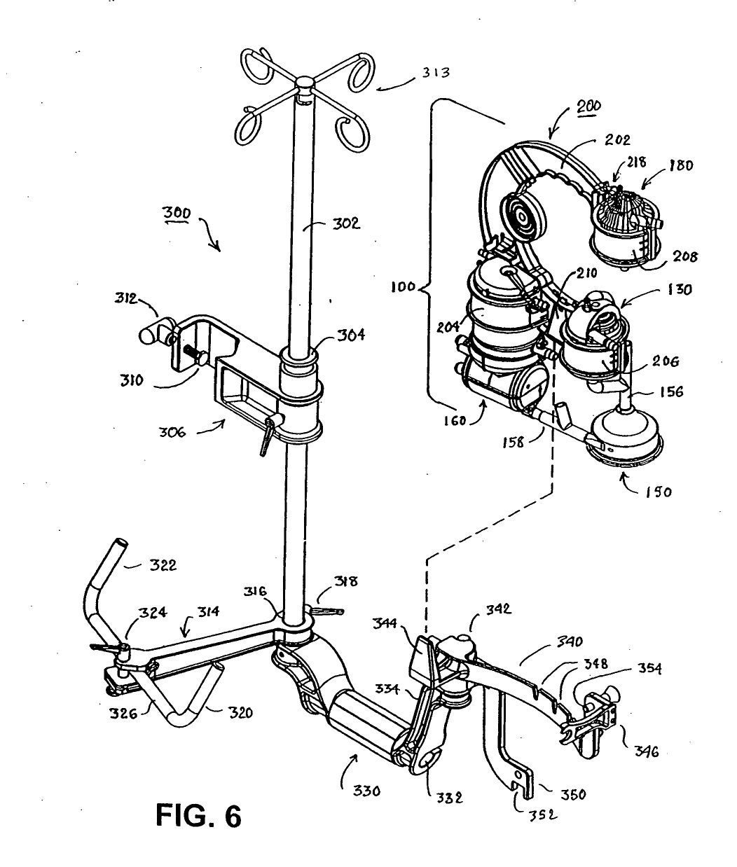

FIGS. 5 and 6 illustrate an extracorporeal blood circuit 100 according to one embodiment of the invention. The extracorporeal blood circuit 100 can include a VARD 130, a centrifugal blood pump 150, an oxygenator 160, and an arterial blood filter 180. The extracorporeal blood circuit 100 is illustrated in FIG. 5 in relation to prime solution holding bags 50 and 52 that drain prime solution into the extracorporeal blood circuit 100 during priming and a sequestering bag 54 adapted to sequester excess prime solution or blood at times during the bypass procedure. The prime solution holding bags 50 and 52 can be intravenous bags including penetrable seals through which spikes can be inserted. The sequestering bag 54 can be supplied with three bag tubes 56, 58, and 60 that have respective Roberts clamps 66, 68, and 70 applied to selectively clamp shut or open the bag tube lumens. For example, the Roberts clamps 66, 68, and 70 may be clamped shut when the sequestering bag 54 is attached to or detached from the extracorporeal blood circuit 100.

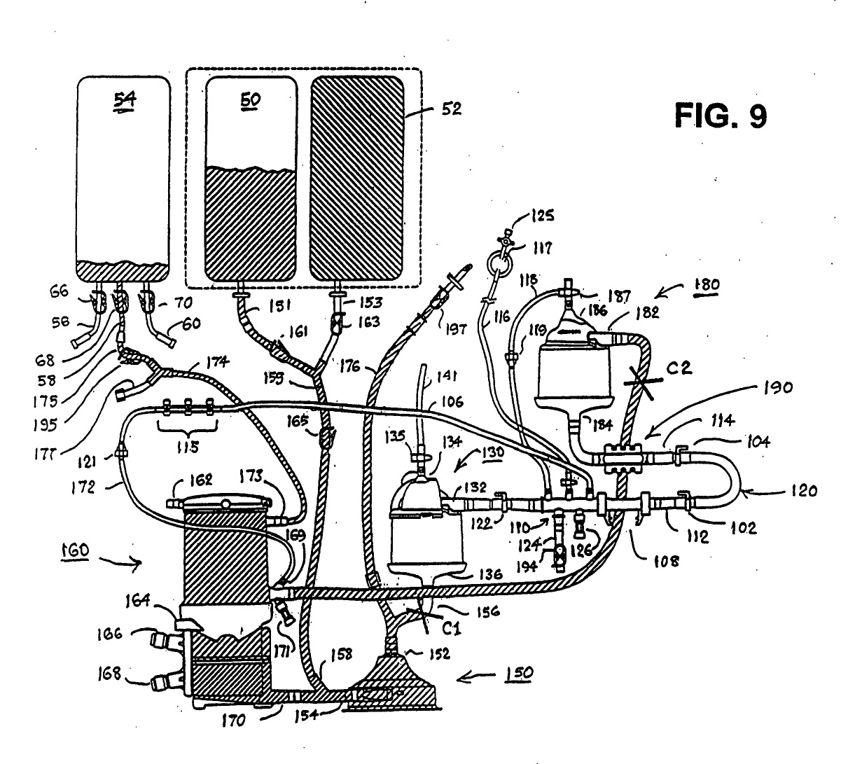

The extracorporeal blood circuit 100 is illustrated in FIG. 5 with a U-shaped, tubular, pre-bypass loop 120 that can be selectively used to connect the arterial blood line 114 with the venous return line 112 during flushing of the extracorporeal blood circuit 100 with CO₂ gas and during priming of the extracorporeal blood circuit 100 with prime solution from prime solution bags 50 and 52 as described further below with respect to FIGS. 9-11. The pre-bypass loop 120 can be coupled to the venous return line 112 by a quick connect connector 102 and to the arterial line 114 by a quick connect connector 104. In one embodiment, the arterial line 114 and venous return line 112 can be formed of 0.375 inch inner diameter polyvinyl chloride tubing.

It will be understood by one of ordinary skill in the art that the pre-bypass loop 120 can be disconnected from the venous and arterial blood lines 112 and 114 after the extracorporeal blood circuit 100 is primed. Table lines extending to venous and arterial cannulae extending into the patient and primed with the patient's blood can then be connected to the respective venous and arterial blood lines 112 and 114 through quick connectors 102 and 104, respectively. Any air that enters the extracorporeal blood circuit 100 during this switching process can be eliminated by the AAR system.

The venous return line 112 can extend from the quick connector 102 through a quick disconnect connector 122 to the inlet 132 of the VARD 130. In one embodiment, a tri-optic measurement cell (TMC) 38 BioTrend® connector 108 having a 0.375 inch inner diameter lumen can be coupled to a utility connector 110 having a 0.375 inch inner diameter lumen and can be interposed in the venous return line 112. The TMC 38 BioTrend® connector 108 may be used to hold a TMC cell (not shown) of the BioTrend™ Oxygen Saturation and Hematocrit System, sold by Medtronic, Inc., to measure blood oxygen saturation and blood hematocrit of venous blood passing through the venous return line 112. The utility connector 110 can support a plurality of standard luer ports and barbed ports.

A venous blood sampling line 106, which can be formed of 0.125 inch inner diameter polyvinyl chloride tubing, can extend between one port of the utility connector 110 to one side of a manifold 115. The manifold 115 can include a rigid tube having a 0.125 inch inner diameter tube lumen and three stopcocks with side vent ports arrayed along the tube.

A venous blood pressure monitoring line 116 can be formed of 0.125 inch inner diameter polyvinyl chloride tubing, can be coupled to a stopcock 196 attached to a luer port of the utility connector 110, and can extend to a pressure isolator 117 and stopcock 125. The pressure isolator 117 of the venous blood pressure monitoring line 116 can include a flexible bladder and can be sized to be attached to a Medtronic® Model 6600 pressure monitor and display box. Venous blood pressure monitoring may be used to optimize kinetic drainage. For example, venous blood pressure that is too high, too low, oscillating, and/or chattering may indicate that the speed of the venous blood pump is incorrect and should be adjusted.

An arterial filter purge line 118, which can be formed of 0.125 inch inner diameter polyvinyl chloride tubing and can include a check valve 119, can extend from a further luer port of the utility connector 110 to the arterial filter purge port 186 of the arterial filter 180. Under operating conditions, a small volume of arterial blood and any air bubbles can be drawn through the arterial filter purge line 118 and check valve 119 from the arterial filter 180 into the venous return line 112. The check valve 119 can prevent reverse flow of venous blood into the arterial filter 180.

In certain cases, it is desirable to provide passive venting of the venous blood in the venous return line 112. A short tube stub 124 can be attached to a barbed port 124 (e.g., with a 0.250 inch inner diameter) extending from the utility connector 110 in order to serve as a vent blood return port. A Roberts clamp 194 can be fitted across the tube stub 124 to be opened or closed when the tube stub 124 is coupled to active or passive venting equipment, e.g., the Gentle Vent passive venting system sold by Medtronic, Inc.

A blood temperature monitoring adapter 126 can extend from the utility connector 110 and can enable insertion of a temperature probe connected with temperature monitoring equipment.

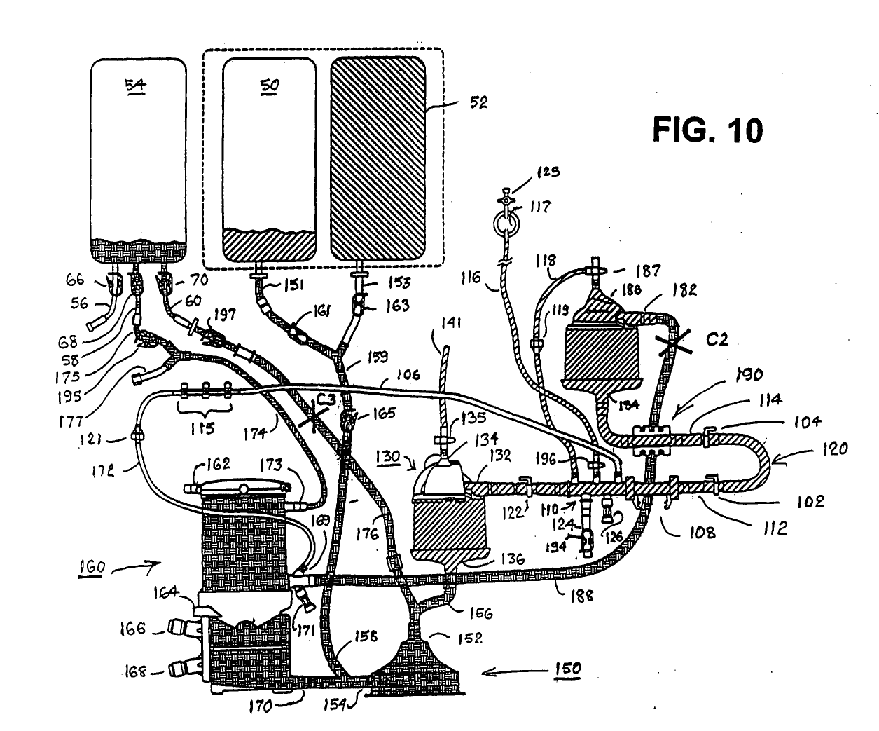

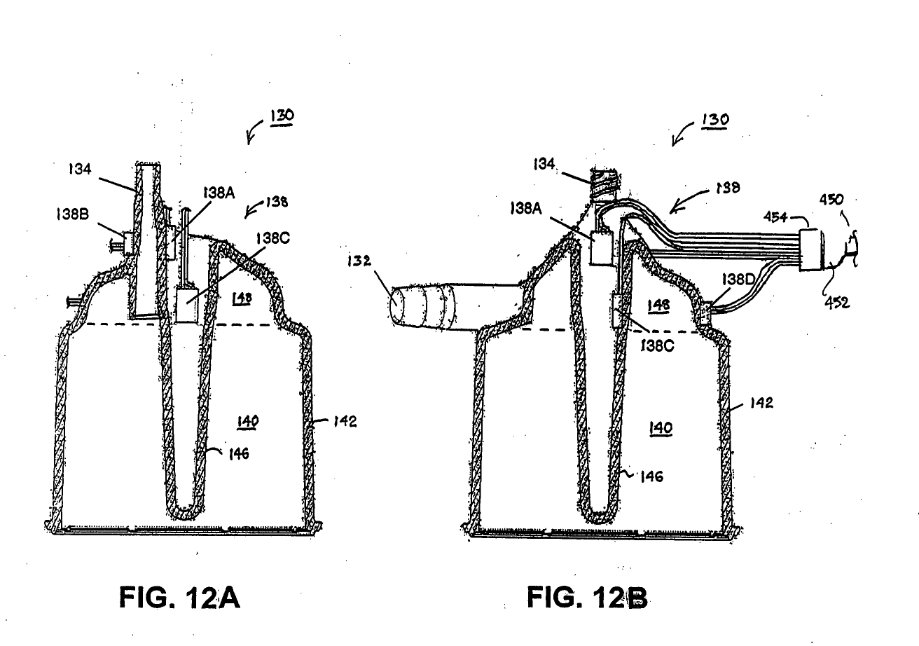

The VARD 130 is described further below with reference to FIGS. 10, 12A, and 12B. In general, air that is entrained in the venous blood drawn through a VARD inlet 132 can be separated from the venous blood within VARD 130 and can accumulate in an upper chamber of the VARD 130. The presence of air can be detected by signals output from air sensors located about the VARD 130, and the air can be evacuated from the chamber.

A venous blood outlet 136 of VARD 130 can be coupled to one branch of a “Y” style segment or line 156, which can be formed of 0.375 inch inner diameter polyvinyl chloride. The trunk of the “Y” style segment or line 156 can be coupled to a blood pump inlet 152 of the centrifugal venous blood pump 150. The blood pump 150 can be adapted to be positioned in use with a drive motor (not shown) that can be selectively operated to draw venous blood through the VARD 130 and pump it into the oxygenator 160.

The venous blood pump 150 can be a centrifugal blood pump, e.g., a BioPump® centrifugal blood pump sold by Medtronic, Inc., that is capable of providing sufficient negative pressure (e.g., to approximately −200 mmHg) for kinetic assisted drainage of venous blood from the patient. Operation of the Bio-Pump® centrifugal blood pump can be controlled by a Bio-Console® drive console sold by Medtronic, Inc. The Bio-Console® drive console can provide electrical energy to drive a reusable pump drive that in turn drives the Bio-Pump® centrifugal blood pump. Exemplary blood pump drive systems are disclosed, for example, in U.S. Pat. Nos. 5,021,048 and 5,147,186.

A fluid infusion line 176, which can be formed of 0.375 inch inner diameter polyvinyl chloride tubing, can be coupled to the other branch of the “Y” style segment or line 156 and can extend to a connection with the tube 60 of the sequestering bag 54, which can be made through a tubing size adapter and Roberts clamp 197. Prime solution can be selectively pumped or drained from the sequestering bag 54 during priming, and blood can be selectively pumped or drained from the sequestering bag 54 during the course of the bypass procedure.

The location of VARD 130 upstream of venous blood pump 150 can provide kinetic assisted venous drainage due to the negative pressure exerted on venous blood by the venous blood pump 150. An AAR system and method can automatically detect and suction off air that collects in a high, quiescent point in the venous line of the extracorporeal blood circuit 100. In one embodiment of the invention, the high point can be within the upper part of VARD 130 adjacent to a purge port 134.

A VARD purge line 141, which can be formed of 0.250 inch inner diameter polyvinyl chloride tubing, can be coupled to the purge port 134 of the VARD 130 through a stopcock 135 and can extend to a vacuum source or pump that can be coupled to the purge line distal end connector 143. A VARD purge line segment 147, which can be formed of silicone rubber, and a vacuum sensor line 145 can be coupled to an AAR controller 400. The VARD purge line 141 or the vent port 134 of the VARD 130 can include a one-way check valve that can prevent air from being pulled into the VARD 130 before the purge line distal end connector 143 is attached to the vacuum source. For example, a check valve 123 can be located at the connection of the VARD purge line 141 and the VARD purge line segment 147. In addition or alternatively, a fluid isolator/filter can be located in the vacuum sensor line 145 at a T-connector 149 to prevent any blood suctioned from the VARD 130 during operation of the AAR system from being suctioned into the vacuum sensor within the AAR controller 400 to which the vacuum sensor line 145 is connected.

The purging vacuum applied through distal end connector 143 may be produced by a pump or it may be produced by connecting the purge line distal end connector 143 directly or indirectly to a vacuum outlet provided in operating rooms. Although not shown in FIG. 5, a liquid trap can be interposed between the purge line distal end connector 143 and the vacuum source or pump to salvage the red blood cells that may be suctioned from the VARD 130 through the VARD purge line 141 and return the blood to the patient. The liquid trap can be a hard-shell venous reservoir, a cardiotomy reservoir, a chest drainage container, or a blood collection reservoir used with the autoLog™ Autotransfusion System sold by Medtronic, Inc. The blood collection reservoir used with the autoLog™ Autotransfusion System has a 40 micron filter and may be mounted onto a mast of the console of the heart-lung machine or other equipment in the operating room to function as a liquid trap. In one embodiment, the vacuum source or pump can be capable of supplying a minimum of about −200 mmHg vacuum, and can be capable of suctioning about 400 ml/mm of air from the liquid trap without the vacuum decreasing below about −180 mmHg.

One end of a trunk of a further “Y” style segment or line 158, which can be formed of 0.375 inch inner diameter polyvinyl chloride tubing, can be coupled to a blood pump outlet 154. One end of a priming line 159, which can be formed of 0.250 inch inner diameter polyvinyl chloride tubing, can be coupled to a side branch of the “Y” style line 158 through a reducing connector. The priming line 159 can extend to branching segments or lines 151 and 153, which can be formed of 0.250 inch inner diameter polyvinyl chloride tubing, that can terminate in spikes that can be inserted into the penetrable openings or seals of the prime solution bags 50 and 52. Roberts clamps 161, 163, and 165 can be fitted over the respective tubing segments or lines 151, 153, and 159 to selectively clamp shut or open the tube lumens during gravity priming of the extracorporeal blood circuit 100. Due to this arrangement, substantially fewer air bubbles can become entrapped in the lines 158 and 159 during priming or operation of the extracorporeal blood circuit 100.

The other branch of the “Y” style tubing segment or line 158 can be coupled to an oxygenator blood inlet 170 of the oxygenator 160 that modulates the temperature of the venous blood and oxygenates the venous blood pumped from the venous blood pump 150. The oxygenator 160 can be a blood oxygenator of the type disclosed U.S. Pat. Nos. 4,975,247, 5,312,589, 5,346,621, 5,376,334, 5,395,468, 5,462,619, and 6,117,390, for example. In one embodiment, the oxygenator 160 includes an AFFINITY® hollow fiber membrane oxygenator sold by Medtronic, Inc.

A blend of oxygen and air can enter the oxygenator 160 through an access port 162 and can exit the oxygenator 160 through an access port 164. Gas exchange between the oxygen and the venous blood entering the oxygenator blood inlet 170 can then take place by diffusion through the pores in the hollow fibers of the oxygenator 160. Thermal energy may be added or removed through a blood heat exchanger, which can be integral with the oxygenator 160. Water can be heated or cooled by a heater/cooler of the heart-lung machine and warmed or chilled water can be delivered to the water-side of the heat exchanger. Water can enter the heat exchanger through a hose (not shown) coupled to a water inlet port 166 and can exit the heat exchanger through a water outlet port 168 and a hose (not shown).

The temperature modulated, oxygenated blood can be pumped out of an oxygenator blood outlet 169 and through an oxygenator outlet line 188, which can be formed of 0.375 inch inner diameter polyvinyl chloride tubing, that can be coupled to an arterial filter inlet 182 of the arterial filter 180. The heated or cooled, oxygenated blood can also be pumped out of a branch of the oxygenator outlet 169 and through an arterial blood sampling line 172 (which can be formed of 0.125 inch inner diameter polyvinyl chloride tubing and can include a check valve 121) that can extend to one input of the manifold 115 for sampling of arterial blood and for drug administration.

A temperature monitoring adapter 171 like adapter 126 can branch from the oxygenator blood outlet 169 to be used to monitor oxygenated blood temperature.

A recirculation/cardioplegia line 174, which can be formed of 0.250 inch inner diameter polyvinyl chloride tubing, can extend from a recirculation port 173 of the oxygenator 160 to a “Y” style connector having two branches 175 and 177. The branch 175 can be coupled to the luer port of line 58 of the sequestering bag 54. A Roberts clamp 195 can be used to open or close the branch 175 of the “Y” style connector coupled to line 58 so that prime solution or oxygenated blood can be selectively pumped into the sequestering bag 54 during the course of priming or performance of the bypass procedure. A second branch 177 of the recirculation/cardioplegia line 174 can include a tube that can be provided with a closed end and can be left intact or cut away so that the recirculation/cardioplegia line 174 can be selectively coupled to a blood cardioplegia source or a hemoconcentrator while the Roberts clamp 195 is closed.

The arterial blood filter 180 may take the form disclosed in U.S. Pat. Nos. 5,651,765 and 5,782,791, for example. In one embodiment, the arterial blood filter 180 can include an AFFINITY® Arterial Filter sold by Medtronic, Inc. The oxygenated blood can be pumped under the pressure exerted by the venous blood pump 150 through the arterial filter inlet 182 through a filter and screen disposed within the arterial blood filter 180 and through an arterial filter outlet 184 into the arterial line 114. Microemboli can be filtered from the oxygenated blood as it passes through the arterial filter 180. Air that is entrained in the oxygenated blood can be separated from the oxygenated venous blood by the screen and can accumulate in an upper chamber of the arterial filter 180 below an arterial filter purge port 186.

The arterial filter purge port 186 can be coupled to a three-way stopcock 187 in an arterial filter purge port 186 that has a branch coupled to an end of arterial filter recirculation line 118. The three-way stopcock 187 can be in an air evacuation position normally that can connect the arterial filter recirculation line 118 with the arterial filter purge port 186. A low volume of arterial blood and air that collects in the upper chamber of the arterial filter 180 below the arterial filter purge port 186 can be drawn by the blood pump 150 through the utility connector 110 and the venous return line 112 into the VARD 130. The difference in pressure between the positive pressure of the oxygenated blood within the chamber of the arterial filter 180 and the negative pressure in the venous return line 112 can draw the blood and air from the chamber of the arterial filter 180 when the venous blood pump 150 is running and the three-way stopcock 187 is moved to the air evacuation position. A check valve 119 in the arterial filter recirculation line 118 can prevent reverse flow of venous blood through the recirculation line 118 when the blood pump 150 is not pumping. The three-way stopcock 187 can be manually moved to a priming position opening the arterial filter chamber to atmosphere to facilitate priming of the extracorporeal blood circuit 100. The arterial filter 180 can be fitted into a receptacle of a circuit support module such that the operator can manually lift and tilt the arterial filter during priming or during the bypass procedure to facilitate evacuation of air observed in the arterial filter 180.

The filtered, oxygenated blood can be returned to the patient as arterial blood through the arterial line 114 coupled to the arterial filter outlet 184 and through a table line fitted to the quick connector 104 and coupled to an arterial cannulae (not shown) or directly to an end of an elongated arterial cannulae extending into the patient's heart. The arterial line 114 can pass through a blood flow transducer connector 190 that can receive and support a Bio-Probe® blood flow transducer sold by Medtronic, Inc., to make arterial flow rate measurements. In normal operation, the Bio-Console® drive console can determine arterial blood flow rate from the output signal of the Bio-Probe® flow probe transducer mounted to blood flow transducer connector 190 to make flow rate measurements of blood flow in arterial line 114 or in oxygenator outlet line 188. Oxygenated, arterial blood flow rate can generally be determined to an accuracy of ±5%.

In some embodiments, the above-described barbed connections and luer connections with lines or tubing do not leak at pressures ranging between +750 mmHg and −300 mmHg. In some embodiments, the barbed connections can withstand pull forces up to 10 lbs linear pull.

Substantially all surfaces of the extracorporeal blood circuit 100 exposed to blood can be blood compatible through the use of biocompatible materials (e.g., silicone rubber, polyvinyl chloride, polycarbonate, or plastisol materials). In one embodiment, the blood contacting surfaces of the extracorporeal blood circuit 100 can be coated with Carmeda® BioActive Surface (CBAS™) heparin coating under license from Carmeda AB and described in U.S. Pat. No. 6,559,132.

In one embodiment, the extracorporeal blood circuit 100 can have operable flow rates of approximately 1-6 liters per minute of blood without producing substantial gas bubbles within the venous blood pump 150 or through the fibers of the oxygenator 160. The extracorporeal blood circuit 100 can be spatially arranged and supported in three-dimensional space by a component organizing and supporting system, which can be positioned at the height of the patient so that the venous return and arterial lines 112 and 114 can be made as short as possible to reduce prime volume.

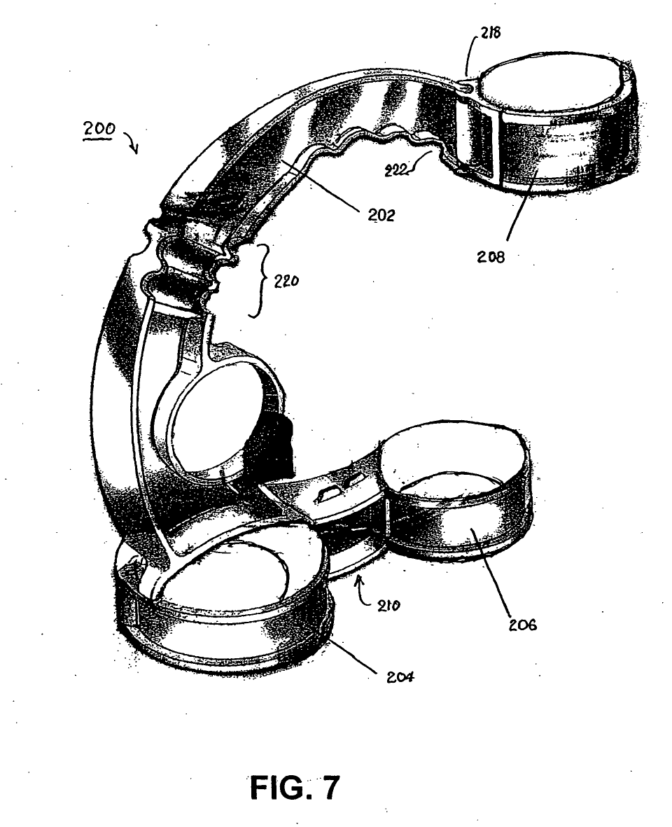

The extracorporeal blood circuit 100 are spatially arranged and supported in three dimensional space, as shown in FIG. 5, by a circuit support module 200 (which can be disposable in some embodiments) and a system holder 300 (which can be reusable in some embodiments), as shown in FIGS. 6-8. Most of the above-described lines and other components interconnecting or extending from the VARD 130, the centrifugal blood pump 150, the oxygenator 160, and the arterial blood filter 180 are not shown in FIG. 6 to simplify the illustration.

The circuit support module 200 can be formed of a rigid plastic material having a C-shaped arm 202 extending between lower snap fittings 204 and 206 and an upper snap fitting 208. A receptacle 210 can be adapted to fit into engagement with the reusable system holder 300. As shown in FIG. 6, the centrifugal blood pump 150 may not be directly supported by the C-shaped arm 202. The “Y” style lines 156 and 158 can couple the centrifugal blood pump 150 to the VARD 130 and the oxygenator 160, which can be supported by the C-shaped arm 202.

The snap fittings 204 and 206 can each include a fixed, concave band formed as part of the C-shaped arm 202 and a separate, U-shaped band. The snap fitting 208 can include a concave band that can be attached to or detached from the C-shaped arm 202 and a separate, U-shaped band. The separate, U-shaped bands can be snapped into engagement with the concave bands to form a generally cylindrical retainer band dimensioned to engage the sidewalls of the oxygenator 160, the VARD 130, and the arterial blood filter 180.

During assembly, the oxygenator 160 can be positioned against the fixed, concave half-band and the U-shaped half-band can be snapped around the oxygenator 160 and into slots on either side of the fixed, concave half-band to entrap oxygenator 160 in the lower snap fitting 204. Similarly, the VARD 130 and the arterial blood filter 180 can be supported and entrapped in the lower and upper snap fittings 206 and 208, respectively. The upper snap fitting 208 encircling arterial blood filter 180 can be detachable at a clip 218 from the C-shaped arm 202. The arterial blood filter 180 and the upper snap fitting 208 can be manually detached at the clip 218, tilted, and then reattached at the clip 218. Air bubbles trapped in the lower portion of the arterial blood filter 180 adjacent the arterial filter outlet 184 can move into the arterial filter purge port 186 to be drawn through the arterial filter purge line 118 into the VARD 130.

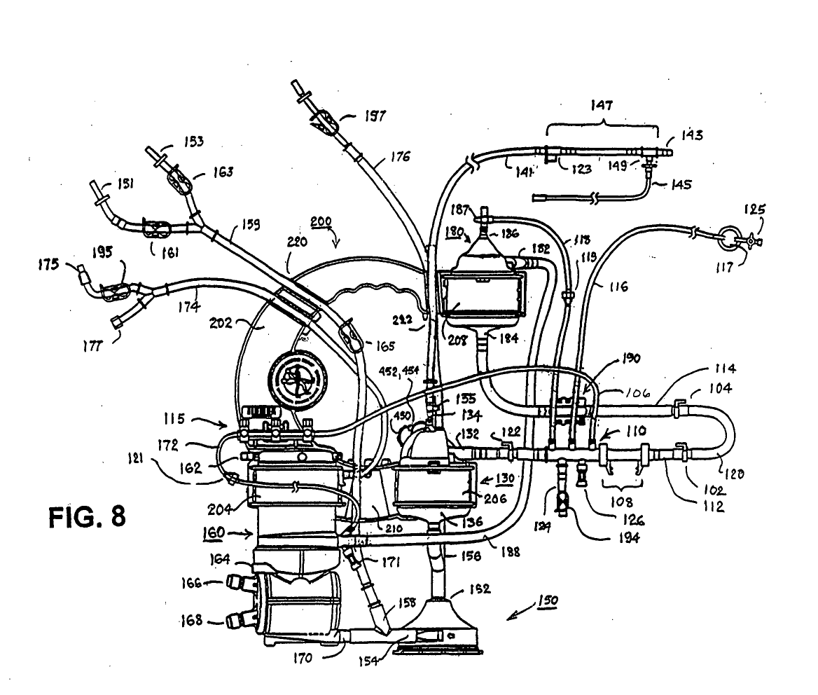

As shown in FIG. 8, lateral raceways 220 and vertical raceways 222 can be provided in the C-shaped arm 202 into which laterally and vertically extending lines, respectfully can be press-fit. The VARD purge line 141 and the fluid infusion line 176 can be extended vertically from the VARD 130 and the branch of the “Y” style line 156, respectively, through the vertical raceway 222. The priming line 159 and the recirculation/cardioplegia line 174 can be extended laterally through the lateral raceways 220.

The circuit support module 200 can maintain proper orientation and positioning of the supported components and the lines extending between or from the supported components. With the circuit support module 200 positioned closer to the patient, shorter lines can be used and can help to minimize the surface area contacted by blood. The oxygenator 160 can be supported by the circuit support module 200, so that the blood pump outlet 154 and the oxygenator blood inlet 170 connected by “Y” style line 158 can be at about the same level below prime solution holding bags 50 and 52, in order to facilitate gravity priming through priming line 159 and retrograde filling of the blood pump 150 and oxygenator 160 with prime solution. The circuit support module 200 can position the VARD 130 above the blood pump 150 and can position the arterial blood filter 180 above the VARD 130, in order to facilitate retrograde priming and movement of air into the arterial filter purge port 186 to be drawn into the VARD 130 and purged.

The circuit support module 200 can allow access for clamping or unclamping the lines or tubing segments or for making connections to the various ports. The circuit support module 200 can allow the venous blood pump 150 to be independently manipulated, e.g., rotated, swiveled, and/or pivoted, with respect to the circuit support module 200 and the system holder 300. The circuit support module 200 can maintain proper positioning and/or alignment of the components and lines of the extracorporeal blood circuit 100 to optimize priming in a relatively short time. In one embodiment, the circuit support module 200 can be transparent to allow sight confirmation of prime solution or blood in the lines and/or other transparent components.

Moreover, the extracorporeal blood circuit 100 and the circuit support module 200 can be assembled as a unit and then attached to the system holder 300 for priming and use during a bypass procedure. A replacement assembly of a extracorporeal blood circuit 100 mounted to a circuit support module 200, as shown in FIG. 8, can be quickly assembled and substituted, if necessary, in a change-out during priming or the bypass procedure.

As shown in FIG. 6, the system holder 300 can include a mast 302 that can extend through a shaft collar 304 of a mast arm assembly 306. The shaft collar 304 can be moved along the mast 302, and the mast arm assembly 306 can be fixed at a selected position by tightening a lever 308. The mast arm assembly 306 can include a U-shaped notch 310 that can be inserted around an upright mast (not shown) of a heart-lung machine console (not shown), and a clamp 312 can be rotated and tightened to hold the mast 302 in a vertical orientation close to the heart-lung machine console. The mast 302 can be provided with an intravenous hanger 313 from which the prime solution holding bags 50 and 52 and the sequestering bag 54 can be hung.

The mast 302 can extend downward from the mast arm assembly 306 and through a collar 316 of an electronics arm assembly 314 that can be moved along the mast 302 and fixed in place by tightening a lever 318. The electronics arm assembly 314 can extend to a cross-bar 326 supporting a right support arm 320 adapted to support an AAR controller 400 and a left support arm 322 adapted to support a pressure monitor and display box (e.g., the Medtronic® Model 6600 pressure monitor and display box sold by Medtronic, Inc.). The support angle provided by the right and left support arms 320 and 322 can be adjusted by loosening a lever 324, pivoting the right and left support arms 320 and 322 to the desired angle, and tightening the lever 324.

The lower end of the mast 302 can be coupled to a laterally-extending support arm assembly 330 that can be formed with a line supporting and routing channel 332. A laterally-extending module arm assembly 340 and a downwardly-extending external drive arm assembly 350 can be mounted to an upward extension 334 of the support arm 330 by a spring lock mechanism 342. A tapered male receiver 344 can extend upward to be received in the downwardly-extending female receptacle 210 of the circuit support module 200 when the extracorporeal blood circuit 100 is mounted to the system holder 300. Line receiving slots 348 can be provided in the laterally-extending module arm assembly 340 for supporting cables for temperature monitoring and a VARD cable 450. The VARD cable 450 can include a cable connector 452 that can be attached to a VARD sensor connector 454, as schematically illustrated in FIG. 12B.

A tri-optic measurement cell (TMC) clip 346 can be fitted to the free end of the laterally-extending module arm assembly 340. The TMC clip 346 can engage a TMC 38 BioTrend® connector 108 into which a TMC cell of a BioTrend® Oxygen Saturation and Hematocrit System can be inserted to measure venous blood oxygen saturation and venous blood hematocrit of venous blood flowing through the venous return line 112 of the extracorporeal blood circuit 100. A cable (not shown) from the TMC cell supported by TMC clip 346 can extend to a BioTrend™ Oxygen Saturation and Hematocrit System.

The Bio-Probe® blood flow transducer sold by Medtronic, Inc. to make blood flow rate measurements through the arterial line can be adapted to be mounted to the laterally-extending module arm assembly 340 at a pin 354. A cable (not shown) can extend from the Bio-Probe® blood flow transducer supported at pin 354 and can extend to a Bio-Probe® blood flow monitor sold by Medtronic, Inc.

An external drive motor for the blood pump 150 can be attached to the free end mount 352 of the external drive arm assembly 350 to mechanically support and drive the blood pump 150 through magnetic coupling of a motor driven magnet in the external drive motor with a magnet of the centrifugal blood pump 150. An adapter can be attached to the free end mount for coupling a hand-cranked magnet with the magnet of the centrifugal blood pump 150 in an emergency situation.

The VARD 130, the centrifugal blood pump 150, the oxygenator 160, and the arterial blood filter 180, as well as the lines and other associated components shown in FIG. 5, can be spatially arranged and supported in three-dimensional space by the circuit support module 200 and the system holder 300, as shown in FIGS. 6-8. The entire assembly can be closely positioned to the heart-lung machine console that operates the drive motor of the centrifugal blood pump 150, that supplies oxygen to the oxygenator 130, and that controls the temperature of the blood or cardioplegia solution traversing the oxygenator 130. The position of the mast arm assembly 306 along the mast 302 can be adjusted to optimally extend the support arm assembly 330 toward and over the patient during the procedure. In some embodiments, the position of the electronics arm assembly 314 along the mast 302 can be adjusted and fixed in place by tightening a lever 318 to optimally position the AAR controller 400 and a Medtronic® Model 6600 pressure monitor and display box for use during the bypass procedure.

Various sensors, lines, and ports can be coupled to other components after the extracorporeal blood circuit 100 is positioned within the circuit support module 200 and mounted to the system holder 300. For example, a reusable VARD sensor cable 450 shown in FIGS. 8 and 14 can extend from the VARD connector 454 laterally through channel 332 to make a connection with an AAR controller 400.

In some embodiments, flushing, priming, and general use of the extracorporeal blood circuit 100 is simplified and made more reliable and efficient. The extracorporeal blood circuit 100 can be flushed with CO₂ gas when the pre-bypass loop 120 is in place after set-up, but before priming, in order to drive out any ambient air. As shown in FIG. 8, the fluid infusion line 176 can be clamped by closing a Roberts clamp 197. As shown in FIG. 14, the VARD purge tubing segment 147 can be fitted into a fluid in-line (FIL) sensor 404, and the T-connector 149 can be fitted into a clip 426 to vertically orient the fluid isolator/filter. The VARD purge tubing segment 147 may not be fitted into a pinch valve 410, so that CO₂ gas can flow through the VARD 130 and the VARD purge line 141 and tubing segment 147 to atmosphere. As shown in FIG. 5, the VARD stopcock 135 can be set to the open position, so that CO₂ gas can flow through the VARD 130 to atmosphere. The arterial filter purge port 186 can be opened to atmosphere by setting the stopcock 187 to the appropriate position, so that CO₂ gas can flow through the arterial filter 180 to atmosphere.

A CO₂ gas delivery line can include a microporous bacteria filter and can be attached to a spike (e.g., a 0.250 inch spike) at the end of one of priming line branches 151 or 153, and the associated Roberts clamp 161 or 163 and the Roberts clamp 165 can be opened. The Roberts clamps 195 and 197 can also be opened. In some embodiments, the CO₂ gas can then be turned on to flow through tubing priming line 159 (e.g., ¼″ polyvinyl chloride) and then through the components and lines of the extracorporeal blood circuit 100 to atmosphere at a flow rate of approximately 2-3 liters per minute. Upon completion, the CO₂ gas can be turned off, and the VARD stopcock 135 can be closed. The priming line branches 151 or 153 can be disconnected from the CO₂ line, and the associated Roberts clamp 161 or 163 can be clamped again.

In one embodiment, the prime volume of the extracorporeal blood circuit 100 can be about 1000 ml or less including a pre-bypass filter (not shown) substituted for the pre-bypass loop 120. In other embodiments, the prime volume of the extracorporeal blood circuit 100, excluding a pre-bypass filter, can be about 900 ml or less. In one embodiment, the extracorporeal blood circuit 100 may be primed using a single one liter intravenous bag 50 of prime solution, e.g., a saline solution. However, in other embodiments, two prime solution bags 50 and 52 can be provided and filled with prime solution for use in initial priming or as required during the bypass procedure.

FIGS. 9-11 illustrate a method of priming the extracorporeal circuit 100 with the bypass circuit 120 in place. The prime solution bags 50 and 52, filled with prime solution, and the empty sequestering bag 54 can be hung on the intravenous hanger 313 (as shown FIG. 6) in preparation for priming. The Roberts clamps 66 and 70 can be left open, as shown in FIG. 9, before the spike ports 56 and 60 are perforated. The branch 177 of the “Y” style connector (which can be attached to the recirculation/cardioplegia line 174 used during cardioplegia) can remain plugged, and the temperature sensor ports 171 and 126 can be sealed by the sensor element. Initially, Roberts clamps 68, 161, 163, 165, 194 and 195 can be closed, and the Roberts clamp 197 can remain open.

The spikes (which can be ¼ inch spikes) of the lines 151 and 153 branching from the priming line 159 (which can also be a ¼ inch in diameter) can be inserted through the penetrable seals of the prime solution bags 50 and 52, respectively. A branch 175 of the “Y” style connector (which can be attached to the recirculation/cardioplegia line 174) can be coupled to the bayonet access port at the free end of the bag line 58 of the sequestering bag 54. The remaining ports and stopcocks can remain as set at the end of the flushing operation. As shown in FIG. 9, tubing clamps (e.g., hemostats) can be applied at about point C1 of the branch of the “Y” style line 156 that can be coupled at its trunk to the blood pump inlet 152 and at about point C2 in the oxygenator outlet line 188, in order to prevent flow of prime solution into the chambers of VARD 130 and arterial blood filter 180, respectively.

The Roberts clamps 161 and 165 can then be opened to gravity fill the pump 150, the oxygenator 160, the fluid infusion line 176, and the oxygenator outlet line 188 with prime solution draining from prime solution bag 50. Filling of the oxygenator outlet line 188 can be assisted by unclamping the tubing clamp at about C2 and applying the tubing clamp again at about C2 when prime solution reaches the arterial filter inlet 182. The Roberts clamp 197 can be closed when the prime solution fills the fluid infusion line 176. One of the Roberts clamps 68 and 195 can be closed, as shown in FIG. 10, when prime solution rises through the recirculation/cardioplegia line 174 and begins to fill the sequestering bag 54. Thus, filling of the oxygenator 160, the pump 150, the fluid infusion line 176, and the recirculation/cardioplegia line 174 can be accomplished in a retrograde fashion to drive air bubbles upward and out of the venous blood pump 150 and oxygenator 160 and the lines coupled therewith, as shown by the cross-hatching in FIG. 9.

As shown in FIG. 10, the spike at the end of the fluid infusion line 176 (e.g., a 0.250 inch spike) can then be inserted into a bayonet port at the free end of bag line 60 extending from sequestering bag 54. The tubing clamp at C1 can be released to allow the prime solution to rise upward through the VARD outlet 136, to fill the VARD 130, and to pass through the VARD inlet 132 into the venous return line 112.

The prime solution can rise upward through the venous return line 112, the utility connector 110, the TMC 38 BioTrend® connector 108, the bypass circuit 120, the arterial line 114 passing through the blood flow transducer 190, and through the arterial filter outlet 184 into the chamber of the arterial filter 180. The check valve 119 can prevent prime solution from rising from the utility connector 110 through the arterial filter purge line 118 to the stopcock 187. The housing of the arterial filter 180 can be transparent so that the retrograde rising prime solution and any air bubbles can be seen. The stopcock 187 can be closed when the prime solution starts to escape the arterial filter purge port 186.

The stopcock 135 can also be opened so that prime solution begins to fill the VARD purge line 141 and can then be closed. At least an upper part of the housing of the VARD 130 can be transparent so that any air bubbles can be seen. The purge line segment 147 can be inserted into the purge line pinch valve 410 to close the purge line segment 147 as the VARD purge line 141 begins to fill with prime solution. The stopcock 135 can be opened, and the stopcocks 196 and 125 can also be opened. The stopcock 125 can then be closed when prime solution rises and fills the venous blood pressure monitoring line 116 and the pressure isolator 117.

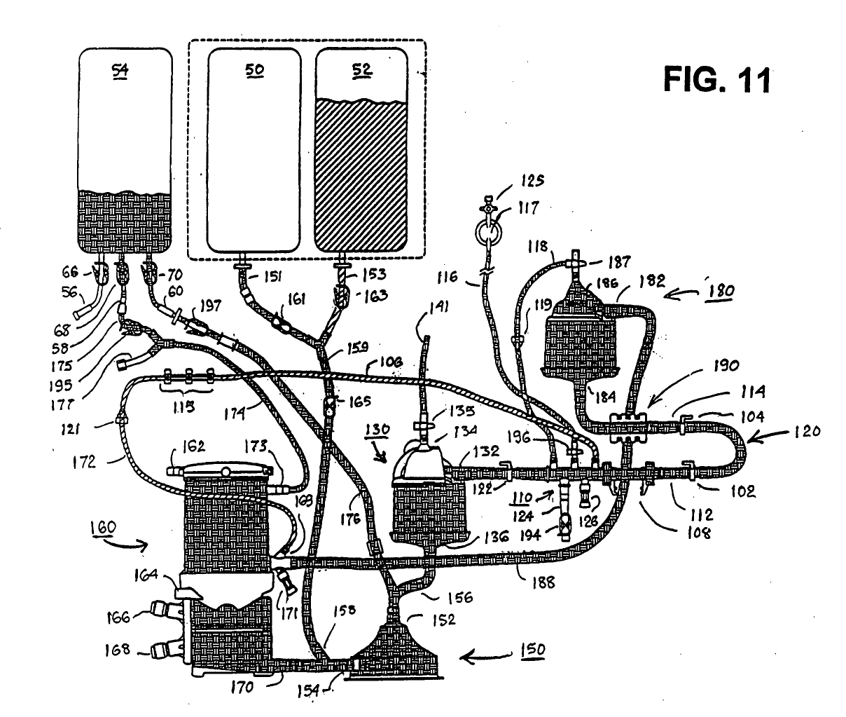

Thus, air can be driven upward and out of the chambers of the VARD 130 and the arterial filter 180 as they are filled with prime solution, as shown in the cross-hatching in FIG. 10. The Roberts clamps 161 and 165 can remain open. As shown in FIG. 11, the tubing clamp that was applied at about C3 can be removed to allow priming fluid to drain from prime solution bag 50 through the priming line 159, the pump 150, and the fluid infusion line 176 into the sequestering bag 54. The sequestering bag 54 can be filled with sufficient prime solution to enable priming of the cardioplegia circuit through the cardioplegia port 56. It may be necessary to open Roberts clamp 163 to drain prime solution from the second prime solution bag 52 in filling sequestering bag 54.

The wall vacuum source can then be coupled to the purge line distal end connector 143 to provide a regulated vacuum (e.g., approximately −215 mmHg) through the VARD purge line 141 when the pinch valve 410 is opened. The VARD sensor cable 450 can be attached to the sensor element connector on VARD 130 and the cable connector 454 on the housing 402 of the AAR controller 400. The Roberts clamp 165 can be closed, the tubing clamp at C2 can be released, and the venous blood pump 150 can be turned on at the minimum flow.

The three stopcocks of sampling manifold 115 can then be set to allow arterial blood flow and air to be drawn by the venous blood pump 150 through the arterial blood sampling line 172, the check valve 121, the sampling manifold 115, the venous blood sampling line 106, and into the utility connector 110. Air can thereby be vented out of the arterial filter purge line 118 and the sampling manifold 115 through the utility connector 110 into the VARD 130 by the venous blood pump 150. The air that accumulates in the VARD upper chamber can then be suctioned out through the line VARD purge line 141 under the action of the VARD controller 400. The arterial filter 180 and a fitting 208 can be detached, inverted, and gently tapped so that the pumped prime solution can move any air in the arterial filter 180 out through the arterial filter outlet 184 and to the VARD 130. The arterial filter 180 can then be reinstalled into the fitting 208 and inspected visually for evidence of any air bubbles that may require repeating of the inverting and tapping steps. The stopcocks of the sampling manifold 115 can then be reset to block flow.

At this point, the extracorporeal blood circuit 100 is primed, and the AAR controller is connected and operational. The pre-bypass loop 120 can be disconnected and table lines can be attached to the quick disconnect connectors 102 and 104. The oxygen lines can be coupled to the access ports 162 and 164 and the water lines can be coupled to the water inlet 166 and water outlet 168 of the oxygenator 160. The AAR controller 400 can be set up to operate the VARD 130.

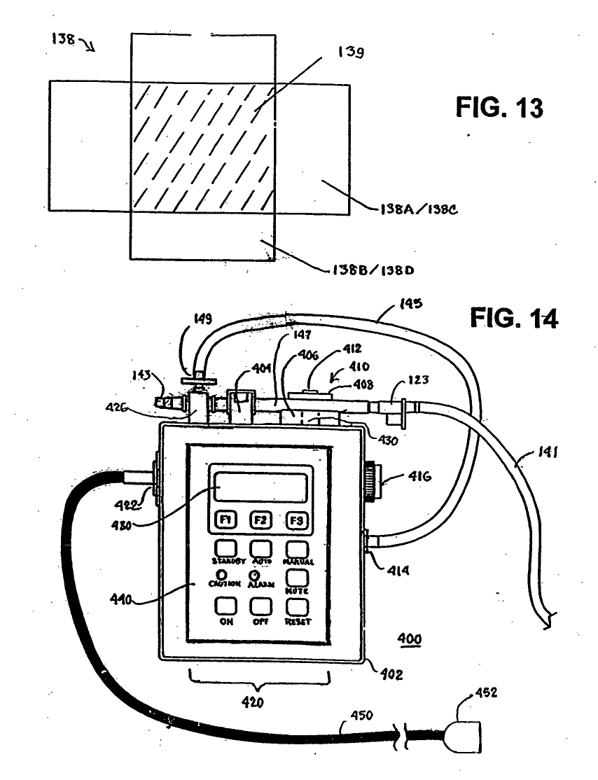

In one embodiment of the invention, an improved AAR system and method can be used to sense and remove air and blood froth from the VARD 130, while removing a minimal amount of liquid blood. The AAR system can include the VARD 130 (as shown in FIGS. 12A, 12B, and 13) which can be controlled by the AAR controller 400 (as shown in FIGS. 14-16). In some embodiments, the AAR system can be capable of removing a continuous stream of air injected into the venous return line 112 at a rate of up to about 200 ml/mm from VARD 130. In one embodiment, the AAR system can handle a maximum rate of air removal of about 400 ml/mm of air and blood froth. In some embodiments, the AAR system is capable of removing a 50 cc bolus of air injected into the venous return line 112 over several seconds from the VARD 130.

The VARD 130 can be a modified conventional arterial blood filter having upper and lower air sensors. For example, the VARD 130 can be a modified AFFINITY® Arterial Filter sold by Medtronic, Inc. Air entrapped in the venous blood can be actively removed by a vacuum applied to the purge port 134 of the VARD 130 through the VARD purge line 141. The VARD 130 can include a housing 142 having a hollow volume displacer 146. The hollow volume displacer 146 can include an inverted cone that can extend down into center of the chamber 140 and can define an annular upper inlet chamber 148. The housing 142 can incorporate components for filtering the venous blood drawn through the housing 142 by blood pump 150 and for detecting and automatically removing air and froth rising to the inlet chamber 148. The lower cap or lower portion of the housing 142 (including the outlet port 136) are not shown in FIGS. 12A and 12B.

The chamber 140 (which can include the inlet chamber 148) of VARD 130 can be filled with blood as the venous blood pump 150 draws venous blood through an upper inlet 144 coupled to venous return line 112 into an inlet chamber 148, through an internally disposed filter element (not shown), and out of the lower VARD outlet 136. A screen or other conventional bubble-trapping device may be inserted in chamber 140 below the inlet chamber 148 to trap air bubbles in the blood stream and cause them to stay in the inlet chamber 148. The VARD 130 can differ from the arterial blood filter 180 in that it can incorporate a sensor array 138. In one embodiment, the sensor array 138 can include four piezoelectric crystal sensor elements 138A, 138B and 138C, 138D, which can be arranged in orthogonally-disposed pairs 138A, 138B and 138C, 138D (as shown in FIGS. 12A, 12B, and 13) in order to sense the level of blood within inlet chamber 148.

In one embodiment of the invention, a first or upper pair of ultrasonic crystals 138A and 138B can be disposed across the vent port 134, and a second or lower pair of ultrasonic crystals 138A and 138B can be disposed across the inlet chamber 148. The crystals 138A and 138C can be bonded onto the exterior surface of the cavity inside the volume displacer 146. The crystals 138B and 138D can be bonded on the exterior surface of the housing extending between the upper portion of the inlet chamber 148 to the vent port 134 and the housing 142, respectively.

In one embodiment, the piezoelectric crystals 138A, 138B and 138C, 138D can be piezoelectric crystal rectangular sheets of a thickness selected to be resonant in the range of 1 to 3 MHz, and specifically about 2.25 MHz, and mounted as shown in FIGS. 12A and 12B. Conductive thin film electrodes can be deposited, plated, or otherwise applied to the major surfaces of the crystals. Conductors can be welded or soldered to the electrodes. Such a piezoelectric crystal can be excited to oscillate in a thickness mode by an RF signal applied, via the conductors and electrodes, across the thickness of the crystal. The resulting mechanical motion of the transmitting crystal can be transmitted though a fluid chamber or conduit. Ultrasonic vibrations emitted by the transmitting crystal can pass through the liquid in the chamber or conduit to impinge upon the receiving crystal. The receiving crystal can vibrate in harmony with the ultrasonic vibrations and can produce an alternating current potential proportional to the relative degree of vibratory coupling of the transmitting and receiving crystals. The degree of coupling of the ultrasonic vibrations can abruptly drop when air is introduced between the transmitting and receiving crystals, and the output amplitude of the signal generated by the receiving crystal can drop proportionally.

In one embodiment, one crystal of each pair 138A, 138B and 138C, 138D can be used as a transmitting crystal, and the other crystal of each pair 138A, 138B and 138C, 138D can be used as the signal receiver. In some embodiments, pairs of crystals (one a transmitter and the other a receiver) are used, rather than a single crystal (as both transmitter and receiver), in order to provide a more robust sensing system. However, some embodiments of the invention can use a single crystal as both the transmitter and the receiver. The presence of liquid or air between the transmitting crystal and the receiving crystal can differentially attenuate the transmitted ultrasonic signal in a manner that can be detected from the electrical signal output by the receiving crystal in response to the ultrasonic signal.

In one embodiment, eight conductors can be coupled to eight electrodes of the piezoelectric crystals 138A, 138B and 138C, 138D. The eight conductors can be extended to a VARD connector 454 (as shown schematically in FIG. 12B), which can be mounted to the VARD housing 142. A distal cable connector 452 of a reusable VARD cable 450 can extend to the AAR controller 400, as shown in FIG. 14. The distal cable connector 452 can be coupled to the VARD connector 454. In one embodiment, the VARD cable 450 can include 10 conductors, and the distal cable connector 452 and the VARD connector 454 can include 10 contact elements. Eight of the cable conductors can be coupled through eight of the mating connector elements with the eight conductive thin film electrodes of the sensor array 138. Two further connector elements of the VARD connector 454 can be electrically in common, and a continuity check can be performed by VARD controller circuitry 460 through the two cable conductors joined when contacting the two connector elements. In this way, a cable or connector failure can be quickly detected and an alarm sounded by the VARD controller 400.

The AAR controller 400 can excite the transmitting crystals and can process the signals generated by the receiving crystals. The AAR controller 400 can include a microprocessor or controller that can use the processed received signals to determine when the liquid level is below the upper crystals 138A, 138B. When the liquid level is below the upper crystals 138A, 138B, the AAR controller 400 can open a pinch valve 410 that normally closes a silicone rubber purge line segment 147. When the pinch valve 410 is open, the VARD purge line 141 can apply suction through the vent port 134 to evacuate the air and froth within the upper inlet chamber 148 below the level of the upper crystals 138A, 138B. The vacuum applied at the vent port 134 can overcome the negative pressure imposed by the venous blood pump 150 within the chamber 148 in order to draw out the accumulated air through the vent port 134. An audible and/or visual warning may be activated to indicate the presence of air within the inlet chamber 148. For example, an audible and/or visual alarm may be activated if liquid, e.g., blood or saline, is not sensed for a particular time period (e.g., approximately five seconds). The warning may continue while air is being removed. When the AAR controller 400 detects liquid between the upper pair of crystals 138A, 138B, the AAR controller 400 can close the pinch valve 410 in order to halt the application of vacuum through the VARD purge line 141.

The lower crystals 138C, 138D, which can be located just above the transition of the main chamber 140 with the inlet chamber 148, can provide a backup to the upper crystals 138A, 138B, should the upper crystals fail. The lower crystals 138C, 138D can also provide a way to detect when the liquid level drops below a minimally acceptable level, even though the AAR controller 400 has opened the pinch valve 410 after detecting air between the upper crystals 138A, 138B. A further distinctive audible and/or visual alarm may be activated if the blood level falls below the lower crystals 138C, 138D. Other embodiments of the invention can include only one set of crystals or even a single crystal positioned to sense the liquid level in the chamber 140. It should also be understood by one of ordinary skill in the art that other types of sensors can be used rather than piezoelectric crystals. Accordingly, the term “sensor(s)” as used herein and in the appended claims refers to piezoelectric crystals or other suitable types of sensors.

In one embodiment of the invention, the crystals 138A, 138B, 138C, 138D can be rectangular in shape and can be arranged so that the long axis of the transmitter crystal 138A, 138C is rotated approximately 90 degrees from the long axis of the receiver crystal 138B, 138D, as shown in FIGS. 12A, 12B, and 13. This configuration can improve transmission overlap at 139 (as shown in FIG. 13) of the transmitted ultrasonic signal to the receiver crystal.

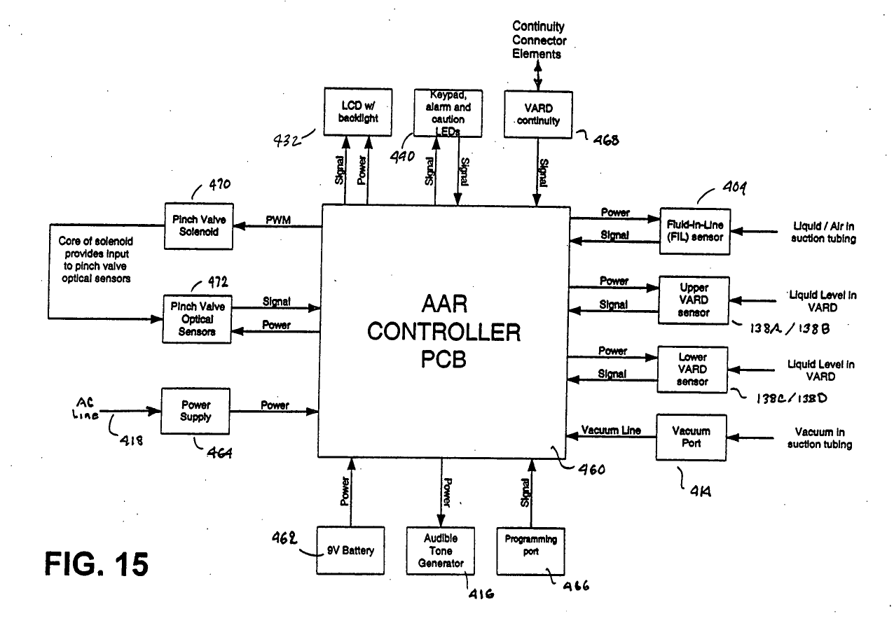

FIGS. 14 and 15 illustrate the AAR controller 400. As shown schematically in FIG. 15, the AAR controller 400 can include AAR controller circuitry 460. The AAR controller circuitry 460 can be powered by an AC line input to a power supply 464, but can also be powered by a back-up battery 462 in case of general power failure or failure of the power supply 464. The AAR controller circuitry 460 can include a microprocessor-based computer operating under control of software stored in memory (e.g., RAM) and can be programmed via a programming port 466. In other embodiments, the AAR controller circuitry 460 can include one or more integrated circuits, programmable logic controllers, or any suitable combination of hardware and software capable of performing one or more of the functions described with respect to FIGS. 16-46.

The AAR controller 400 can include a clamp (not shown) on the rear side of a housing 402, and the clamp can be adapted to be attached to the left support arm 322 of the system holder 300 (as shown in FIG. 6). After attachment, a user interface 420 (including a display 430 and a control panel 440) can be positioned outward for reading the displayed text and/or warning lights and for use of controls on the control panel 440.

A clip 426, a fluid in-line (FIL) sensor 404, and a pinch valve 410 can be disposed on the housing 402. The FIL sensor 404 can include a lid, which can extend across a notch so that the cross section of the notch is substantially constant when the lid is closed. The lid can be opened, the VARD purge line 141 can be extended laterally across the oxygenator 160, a first section of the VARD purge line segment 147 can be fitted into the notch of the FIL sensor 404, and the lid can be closed. A T-connector 149 can be fitted into the clip 426 with the vacuum sensor line 145 extending vertically.

The pinch valve 410 can include upper and lower members 406 and 408 that can define a slot within which a second section of the VARD purge line segment 147 can be positioned. A pinch rod 430 can extend upward from within the housing 402. The pinch rod 430 can be under spring tension and can extend transversely into the slot between the upper and lower members 406 and 408. The pinch rod 430 can be moved downward out of the slot when a mechanical release button 412 is pressed, in order to insert the second section of the VARD purge line segment 147 into the slot. The pinch rod 430 can then compress the second section of VARD purge line segment 147 upon release of the mechanical release button 412. The pinch rod 130 can be retracted by again depressing the mechanical release button 412 or by operation of a solenoid controlled by the AAR controller 400.

In some embodiments, the tubing of the purge line segment 147 inserted into the slot can be constructed of a soft, biocompatible material having a suitable durability and resilience (e.g., silicone rubber tubing). In one embodiment, the silicone rubber tubing of the purge line segment 147 can have a 0.250 inch inner diameter and a 0.375 inch outer diameter, and the silicone rubber tubing can have sufficient resilience to restore the lumen diameter to at least 3/4 of its nominal lumen diameter upon retraction of the pinch rod 430.

The distal end of the vacuum sensor line 145 can be attached to a vacuum sensor input 414 on the housing 402, as shown in FIG. 14. An audible tone generator 416 can be mounted to the housing 402. An AC power cord 418 can be attached to the housing 402. The VARD sensor cable 450 (in one embodiment, including the eight conductors attached to the eight surface electrodes of the piezoelectric crystals 138A, 138B, 138C and 138D and the two continuity checking conductors) can extend between the cable connector 452 and the cable connector 422 on the housing 402. In some embodiments, the purge line segment 147 fitted into the FIL sensor 404 and a pinch valve 410 can be at the same level as the VARD purge port 134. The height of the AAR controller 400 can be adjusting by moving the electronics arm assembly 314 along the mast 302.

The pinch rod 430 can be axially aligned with and coupled to a solenoid core that can move downward into the housing 402 when the solenoid coil is energized. A solenoid driver 470 (as shown schematically in FIG. 15) can be selectively actuated by the AAR controller circuitry 460 to drive the pinch rod 430 downward, overcoming the biasing force of a spring. In one embodiment, pinch valve sensors 472 (as shown schematically in FIG. 15) can be provided within the housing 402 to determine the position of the downwardly-extending pinch rod 430 or the solenoid core coupled to the pinch rod 430. The pinch rod sensors 472 can provide output signals to indicate whether the pinch rod 430 is in an upper closed position, a lower open position, or in a fault position between the upper and lower positions. The output signals can confirm that the pinch rod 430 has moved between positions in response to the applied appropriate command, or that the pinch rod 430 is malfunctioning.

As shown in FIG. 14, a user interface 440 can include controls, such as soft keys. The soft keys can include an “ON” key and an “OFF” key that can be depressed to power up and power down, respectively, the controller circuitry and sensors. A “RESET” key can be depressed to reset the controller signal processor. A “CAUTION” light (e.g., a yellow LED) and an “ALARM” light (e.g., a red LED) can be lit when the signal processor determines certain respective caution and alarm conditions. Respective audible caution and alarm tones can be emitted by an audible tone generator 416. A “MUTE” switch can be depressed to silence the audible tones. “STANDBY” and “AUTO” buttons can be depressed to initiate respective standby and automatic operating modes. Manual depression of a “MANUAL” soft key can open the pinch valve 410 for as long as the “MANUAL” soft key remains depressed or for a particular time period. Function keys F1, F2, and F3 can be depressed in response to a message displayed on the display 432.

When the AAR controller is operating in an automatic mode, the solenoid driver 470 can be actuated automatically when air is detected in the VARD chamber 148 and/or when other conditions are met. The solenoid driver 470 can also be actuated in response to a user-initiated command. The pinch rod 430 can be released to open the lumen of the VARD purge line segment 147 by depressing mechanical release button 412.

The purging operation in the automatic mode can be dependent upon a number of conditions and sensor input signals, such as one or more of those described as follows. The output signal of the upper crystals 138A, 138B (or the lower crystals 138C, 138D) can indicate that air is present in the VARD upper chamber 148. A vacuum threshold level can be met by the vacuum in the vacuum line segment 147, as measured through vacuum sensor line 145 and T-connector 149 by the vacuum sensor coupled to vacuum sensor input 414. The output signal of the FIL sensor 404, which is proportional to the amount of fluid in the vacuum line segment 147, should not exceed an FIL signal threshold. In general, the operating states of a number of components and sensors can be monitored, and the operating states can determine whether the automatic mode can be performed using the piezoelectric crystals 138A, 138B, 138C and 138D.

The output signals from the position sensors 472 can confirm whether the pinch rod 430 is in a fully-open or a fully-closed position. Positions other than a fully-open or a fully-closed position may be considered error states and an audible and/or visible alarm may be activated. The pinch valve 410 may be electrically operated, pneumatically operated, or manually operated in case of a power failure.

The AAR controller 400 can perform a Self Test of one or more components. In one embodiment, the following five components can undergo the Self Test:

Display 432: A liquid crystal display (LCD) can be solid a particular time period (e.g., about two seconds), followed by a display of the version of the installed software.

Indicators: The CAUTION light and/or the ALARM light can flash momentarily.

Audible Indicator: A single “chirp” with a delay (e.g., about one second) between sounds can occur for several seconds.

Pinch Valve 410: The pinch valve solenoid 470 can open and close the pinch valve slot to verify proper operation.

Battery 462: The power level in the battery 462 (e.g., a 9 Volt batter) can be evaluated.

In one embodiment, upon successful completion of the Self Test, the display 432 can indicate “NO ERROR DETECTED” and the operating algorithm can automatically switch to a Standby Mode. The appropriate corrective action can be taken if an error is indicated on the display 432 upon completion of the Self Test or during the Standby Mode. Priming of the extracorporeal blood circuit 100 can then be commenced, and the AAR system can be used when the blood pump 150 is performing the priming function.

The AAR system can then be used in manual or automatic operating modes to detect and remove air in the VARD 130. In one embodiment, in either the manual or automatic operating modes, the Caution message “AIR IN VARD” can appear on the display 432 when air is detected between the upper crystals 138A, 138B. In addition or alternatively, the CAUTION light can flash and/or a repeating, audible tone can be emitted by the tone generator 416 when air is detected and/or being removed.

In some embodiments, the manual mode can override the automatic mode, e.g., in order to compensate for an error or a low-battery state. For example, the pinch valve 410 can be opened (by the pinch rod 430 being retracted from the slot) by depressing the mechanical button 412.

If the AAR controller circuitry 460 is powered by line power, the user can also manually evacuate the air by depressing the MANUAL key on the user interface 440. Depressing the MANUAL key can open the pinch valve 410 and can allow the vacuum source coupled to nozzle 143 to remove air from the VARD 130 through the VARD purge line 141. Once air has been removed from the VARD 130, the user can release the MANUAL key to close the pinch valve 410. When the MANUAL key is depressed and the AAR controller circuitry 460 is functioning, the display 430 can indicate “VALVE OPEN.” Depressing the MANUAL key can over-ride the automatic response to an output signal of the FIL sensor 404 detecting fluid in the tubing segment 147 and can prevent the automatic closing of the pinch valve 410. The message “AIR IN VARD” can automatically clear from the display 432, and the display 432 can reverts to the Standby Mode display. The CAUTION light can stop flashing and/or the audible tone can stop being emitted.

The automatic mode can be initiated by depressing the AUTO key on the user interface 440. In the automatic mode, the pinch valve 410 can be automatically opened in some embodiments, as long as air is detected between the upper crystals 138A, 138B, as long as the output signal of the FIL sensor 404 indicates fluid is in the tubing segment 147, and/or as long as other operating conditions are satisfied. For example, if the AAR controller 400 is running on battery back-up power, the pinch valve 410 may not open automatically when the upper crystals 138A, 138B detect air in the VARD 130. The display 432 can indicate “OPEN THE VALVE.” In this case, pressing the MANUAL key will not open the pinch valve 410. The CAUTION light can flash and repeating audible tones can sound. To open the pinch valve 410 manually, the user can depress the mechanical button 412.

The automatic mode screens for the display 432 of one embodiment of the invention are shown and described with respect to FIGS. 16-18. A user can first ensure that the wall vacuum regulator is adjusted to −225 mmHg. The user can press the AUTO key. The AAR controller 400 can transition from the Standby Mode to the Automatic Mode, as indicated in FIG. 16 (Automatic Mode, Normal Operation).

A Caution condition can occur in the Automatic Mode when the upper crystals 138A, 138B in the VARD 130 detect air. The AAR controller 400 can command the pinch valve 410 to open automatically to evacuate the air in the VARD 130. As shown in FIG. 17 (Automatic Mode, Caution State), the display 432 can read “AIR IN VARD.” The CAUTION light can flash and the repeating, audible tone can sound.

After the air in the VARD is removed and the upper crystals 138A, 138B detect fluid, the AAR controller 400 can command the pinch valve 410 to automatically close. The display 432 indication of “AIR IN VARD” can clear. The CAUTION light can stop flashing and the audible tones can stop. In the Battery Back-Up Mode, the pinch valve 410 may not open automatically when the upper crystals 138A, 138B detect air. Pressing the MANUAL key may not open the pinch valve 410. However, a user can press the mechanical button 412 on top of the pinch valve 410 to evacuate any accumulated air in the VARD 130.

In the AAR controller 400 is running on battery back-up and air is detected in the VARD 130, the display 432 can read “OPEN THE VALVE,” as shown in FIG. 18 (Automatic Mode, Caution State in Battery Back-Up). The CAUTION light can flash and repeating audible tones can sound. A user can press the mechanical button 412 on top of the pinch valve 410 to remove any accumulated air in the VARD 130. Once air is removed, the user can release the mechanical button 412. When the upper crystals 138A, 138B detect fluid, the display 432 indication of “OPEN THE VALVE” can clear. The CAUTION light can stop flashing and the audible tones can stop.

The troubleshooting screens for the display 432 and the responses taken are shown in FIGS. 19-46. FIGS. 19-25 illustrate screens for Self Test Mode Corrective Action Procedures. FIG. 19 illustrates the screen for a self test Cyclical Redundancy Check (CRC) failure, corresponding to the following Message, Condition, and Corrective Action information:

Message: CRC Failure—FIG. 19

Condition: The Cyclical Redundancy Check (CRC), indicating a general software failure.

Corrective Action: Press RESET. If failure persists, a user can call a local Technical Support representative. Use a back-up AAR controller 400.

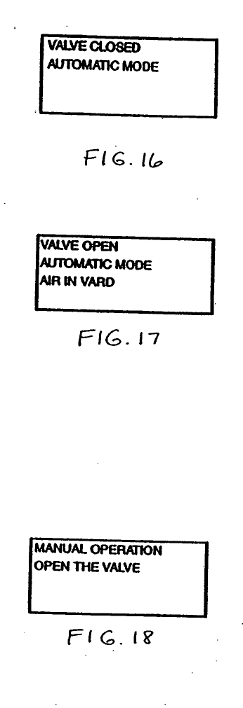

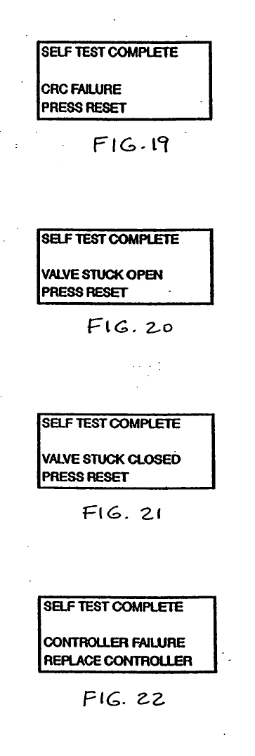

FIG. 20 illustrates the screen for a self test pinch valve stuck open failure, corresponding to the following Message, Condition, and Corrective Action information:

Message: Valve Stuck Open—FIG. 20

Condition: The Pinch valve is stuck in the OPEN position when it should be closed.

Corrective Action: Press RESET. If failure persists, a user can call a local Technical Support representative. Use a back up AAR controller 400.

FIG. 21 illustrates the screen for a self test pinch valve stuck closed failure, corresponding to the following Message, Condition, and Corrective Action information:

Message: Valve Stuck Closed—FIG. 21

Condition: The pinch valve is stuck in the CLOSED position when it should be open.

Corrective Action: Press the RESET key. If failure persists, a user can call a local Technical Support representative. Use a back-up AAR controller 400.

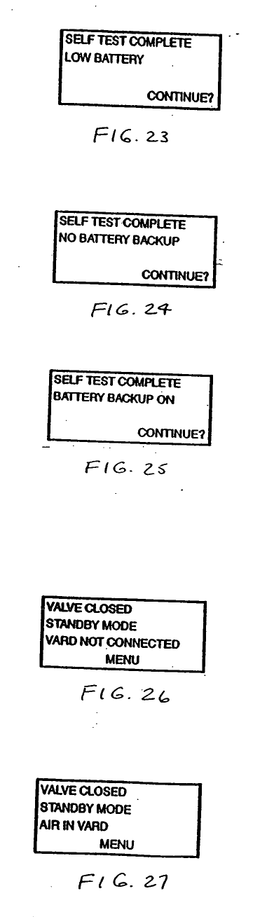

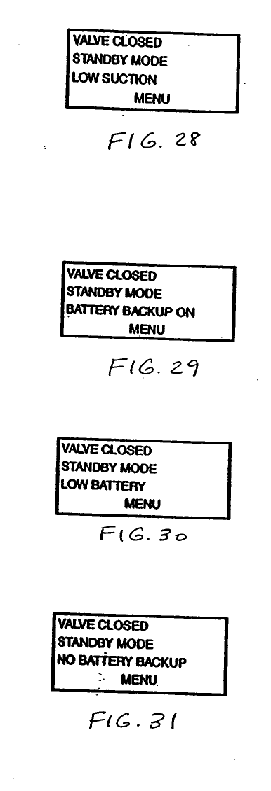

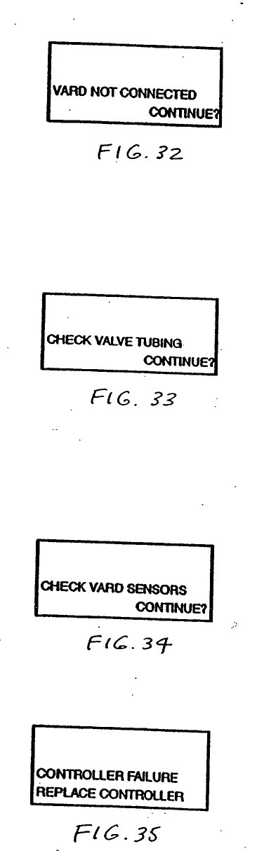

FIG. 22 illustrates the screen for a self test pinch valve general failure, corresponding to the following Message, Condition, and Corrective Action information:

Message: Controller Failure—FIG. 22

Condition: The pinch valve failed the software test.

Corrective Action: Press the RESET key. If failure persists, a user can call a local Technical Support representative. Use a back-up AAR controller 400.

FIG. 23 illustrates the screen for a self test low battery message, corresponding to the following Message, Condition, and Corrective Action information:

Message: Low Battery—FIG. 23

Condition: Battery power is below minimum specifications.

Corrective Action: Replace battery per the IFU or press the F3 key to transition to Standby Mode.

FIG. 24 illustrates the screen for a self test battery failure, corresponding to the following Message, Condition, and Corrective Action information:

Message: No Battery Back-Up—FIG. 24

Condition: Self Test failed to detect a functional battery back-up circuit.

Corrective Action: Press the F3 key. If failure persists, a user can call a local Technical Support representative. Use a back-up AAR controller 400.

FIG. 25 illustrates the screen for a self test battery back-up on state, corresponding to the following Message, Condition, and Corrective Action information:

Message: Battery Back-Up On—FIG. 25

Condition: The AAR controller 400 was started in the Battery Back-up Mode.

Corrective Action: Plug the AAR controller 400 into an AC outlet.

FIGS. 26-31 illustrate screens for Standby Mode Corrective Action Procedures. A user can initiate the appropriate corrective action if any of the Caution messages in FIGS. 26-31 appear on the display 432 during the Standby Mode. If necessary, the user can press the RESET key. FIG. 26 illustrates the screen for a standby mode, VARD not connected state, corresponding to the following Message, Condition, and Corrective Action information:

Message: VARD Not Connected—FIG. 26

Condition: AAR controller 400 is not detecting the cable connection between the AAR controller 400 and the VARD 130.

Corrective Action: Connect the cable, or replace the cable.

FIG. 27 illustrates the screen for a standby mode, air in VARD state, corresponding to the following Message, Condition, and Corrective Action information:

Message: Air In VARD—FIG. 27

Condition: Air is present in the VARD 130 at the level of upper crystals 138A, 138B.

Corrective Action: Prime the VARD 130, or press the MANUAL key on the user interface 440 to withdraw air from the VARD 130.

FIG. 28 illustrates the screen for a standby mode, low suction state, corresponding to the following Message, Condition, and Corrective Action information:

Message: Low Suction—FIG. 28

Condition: There is insufficient negative pressure being detected by the AAR controller 400.

Corrective Action: Connect the suction monitoring line to the pressure sensor. Connect the suction source. Increase vacuum.

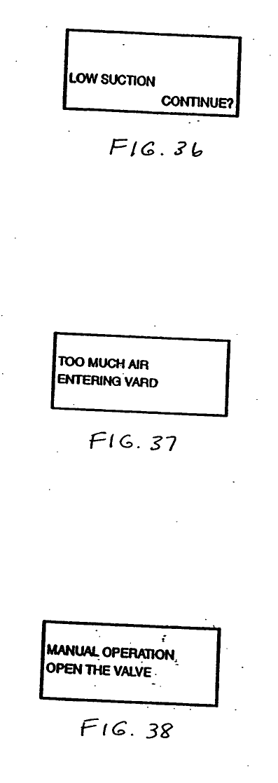

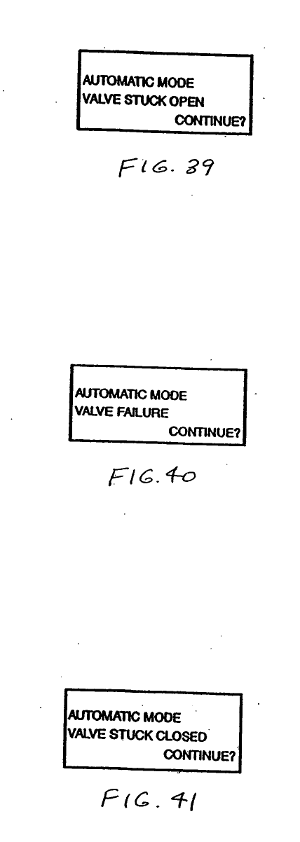

FIG. 29 illustrates the screen for a standby mode, battery back-up state, corresponding to the following Message, Condition, and Corrective Action information:

Message: Battery Back-Up On—FIG. 29

Condition: The AAR controller 400 is being powered by the 9-volt long life alkaline battery.

Corrective Action: Confirm the AAR1000 is plugged into a functioning 120-volt AC receptacle, or replace the power cable, or call a local Technical Support representative and use a back-AAR controller 400.

FIG. 30 illustrates the screen for a standby mode, low-battery state, corresponding to the following Message, Condition, and Corrective Action information:

Message: Low Battery—FIG. 30

Condition: Battery power is below minimum specification in Battery Back-up Mode.

Corrective Action: Replace the battery per the instructions.

FIG. 31 illustrates the screen for a standby mode, battery failure, corresponding to the following Message, Condition, and Corrective Action information:

Message: No Battery Back-Up—FIG. 31

Condition: The AAR controller 400 failed to detect a functional battery back-up circuit.

Corrective Action: Call a local Technical Support representative and use a back-up AAR controller 400.

FIGS. 32-46 illustrate screens for Automatic Mode Corrective Action Procedures. FIGS. 32-36 illustrate screens for a Transition Mode. The messages shown in FIGS. 32-36 may appear on the display 432. The AAR controller 400 may not convert (“Transition”) to the Automatic Mode until the condition in the display 432 is corrected or the F3 key is pressed. FIG. 32 illustrates the screen for a transition mode, VARD not connected state, corresponding to the following Message, Condition, and Corrective Action information:

Message: VARD Not Connected—FIG. 32

Condition: Software is not detecting the cable connection between the AAR1000 and the VARD.

Corrective Action: Connect the cable, or replace the cable, or press F3 to return to Standby Mode.

FIG. 33 illustrates the screen for a transition mode, check tubing state, corresponding to the following Message, Condition, and Corrective Action information:

Message: Check Valve Tubing—FIG. 33

Condition: The AAR controller 400 is not detecting the silastic tube in the pinch valve.

Corrective Action: Insert silastic tube in the pinch valve, or reposition the silastic tube in the pinch valve, or press F3 to return to standby Mode, or call a local Technical Support representative and replace with a back-up AAR controller 400.

FIG. 34 illustrates the screen for a transition mode, check tubing state, corresponding to the following Message, Condition, and Corrective Action information:

Message: Check VARD Sensors—FIG. 34

Condition: The software is detecting an improper signal from the ultrasonic sensors on the VARD.

Corrective Action: Disconnect and reconnect the VARD sensor cable. Replace the cable, or press F3 to return to Standby Mode, or call a local Technical Support representative and replace with a back-up AAR controller 400.

FIG. 35 illustrates the screen for a transition mode, circuit failure, corresponding to the following Message, Condition, and Corrective Action information:

Message: Controller Failure—FIG. 35

Condition: The pinch valve failed the software test

Corrective Action: Press the RESET key. If failure persists, call a local Technical Support representative. Use a back-up AAR controller 400.

FIG. 36 illustrates the screen for a transition mode, low suction state, corresponding to the following Message, Condition, and Corrective Action information:

Message: Low Suction—FIG. 36

Condition: There is insufficient negative pressure being detected by the AAR controller 400.

Corrective Action: Attach the suction monitoring line to the sensor, and ensure the connections are secure, or connect the wall vacuum source, or increase the vacuum, or after correcting the problem, press F3 to enter Automatic Mode. Initiate Cardiopulmonary Bypass. Monitor the Resting Heart™ Module and the AAR controller 400 for operational charges that may cause a Caution condition or Alarm condition to occur.

FIGS. 37-46 illustrate screens for Alarm Conditions. Alarm condition in the Automatic Mode can occur when the lower crystals 138C, 138D in the VARD 130 detect air. The AAR controller 400 can command the pinch valve 410 to open automatically to evacuate the air in the VARD 130. The ALARM light can flash and two rapid repeating, audible tones can sound. Pressing the MUTE key can only silence the audible tones for 15 seconds. The messages shown in FIGS. 37-46 may occur at the time of an Alarm condition and warrant the immediate intervention and corrective action on the part of the user.

When too much air is entering the VARD 130 (FIG. 37), the user can perform the following procedure: Immediately reduce pump flow. This will improve the efficiency at which the suction source removes air for the VARD 130. Check that the vacuum source is at −225 mmHg. Check for loose fittings or disconnects in the venous circuit proximal to the VARD 130. Confirm with the surgeon that the venous catheter is properly positioned and the right atrial purse strings at the cannulation site are secure. As the blood level in the VARD 130 rises above the lower and upper crystals, the Alarm message will clear, the ALARM light will stop flashing, and the audible tone will stop. Resume normal blood flow once the air is totally removed from the VARD 130.

The user can perform the following procedure to open the pinch valve 410 (FIG. 38): In the Battery Back-up Mode, the pinch valve 410 will not open automatically when the lower crystals 138C, 138D detect air. Pressing the MANUAL key will not open the pinch valve 410. Press the mechanical button 412 on the top of the pinch valve 410 to evacuate any accumulated air in the VARD 130. Immediately reduce pump flow if necessary. This will improve the efficiency at which the suction source removes air from the VARD 130. Press and hold the mechanical button 412 on the top of the pinch valve 410. This will open the pinch valve 410 to evacuate air in the VARD 130. The pinch valve 410 will stay open as long as the mechanical button 412 is being pressed. Release the mechanical button 412 when air has been sufficiently removed from the VARD 130. Check for loose fittings or disconnects in the venous circuit proximal to the VARD 130. Confirm with the surgeon that the venous catheter is properly positioned and the right atrial purse strings at the cannulation site are secure. Repeat pressing the mechanical button as necessary to evacuate air from the VARD 130. As the blood level in the VARD 130 rises above the lower crystals 138C, 138D, the AAR controller 400 will revert to the Caution condition. Resume normal blood flow once the air is totally removed from the VARD 130. If there has been no disruption in the hospital electrical power, check the AC power cord for a disconnection.

The user can perform the following procedure when the pinch valve 410 is stuck open (FIG. 39): Press the F3 key to clear the error condition. Observe the VARD 130 for the presence of air. If there is no visual evidence of air in the VARD 130, clamp the VARD purge line 141 with a hemostat proximal to the one-way valve 123 at the inlet of the pinch valve 410. If there is visual evidence of air in the VARD 130, continue to allow the wall vacuum source to evacuate the air. Once the air is removed and the message persists, clamp the VARD purge line 141 with a hemostat proximal to the one-way valve 123 at the inlet to the pinch valve 410. Closely monitor the circuit for the appearance of air in the venous line 112 and the VARD 130. Manually open and close the hemostat on the VARD purge line 141 to evacuate air as necessary. If problem persists, call a local Technical Support representative and use a back-up AAR controller 400.

The user can perform the following procedure when the pinch valve 410 fails (FIG. 40): Press the F3 key to clear the error condition. Inspect the VARD 130 for the presence of air. Visually confirm the position of the pinch valve 410. If there is no visual evidence of air in the VARD 130 and the pinch valve 410 is open, manually clamp the VARD purge line 141 with a hemostat proximal to the one-way valve 123. If the pinch valve 410 is closed, manually clamp the VARD purge line 141 with a hemostat proximal to the one-way valve 123, press down on the mechanical button 412 on the top of the pinch valve 410 and remove the silastic tube from the pinch valve 410. Closely monitor the venous line 112 and the VARD 130 for entrapment of air. Unclamp and clamp the hemostat at the VARD purge line 141 as necessary to evacuate air. Call a local Technical Support representative and use a back-up AAR controller 400.

The user can perform the following procedure when the pinch valve 410 is stuck closed (FIG. 41): Press the F3 key to clear the error condition. Press the MANUAL key on the front panel to open the pinch valve 410. If the MANUAL key fails to operate, manually clamp the VARD purge line 141 with a hemostat proximal to the one-way valve 123, press down on the mechanical button 412 on top of the pinch valve 410, and remove the silicone tube from the pinch valve 410. Closely monitor the venous line 112 and the VARD 130 for air entrapment. Unclamp and clamp the hemostat at the VARD purge line 141 as necessary to evacuate air. Call a local Technical Support representative and use a back-up AAR controller 400.

The user can perform the following procedure during a system failure when blood is being removed (FIG. 42): Press the F3 key to clear the error condition. Immediately clamp the VARD air removal line 141 proximal to the one-way valve 123. If the pinch valve 410 is closed, manually clamp the VARD purge line 141 with a hemostat proximal to the one-way valve 123, press down on the mechanical button 412 on the top of the pinch valve 410 and remove the silastic tube form the pinch valve 410. Closely monitor the venous line 112 and the VARD 130 for air entrapment. Unclamp and clamp the VARD air removal line 141 as necessary to evacuate air. Call a local Technical Support representative and use a back-up AAR controller 400.

The user can perform the following procedure during a VARD sensor failure (FIG. 43): Press the F3 key to clear the error condition. Immediately manually clamp the VARD line 141 with a hemostat proximal to the one-way valve 123. Press the mechanical button 412 on top of the pinch valve 410 and remove the silicone tube from the pinch valve 410. Closely monitor the venous line 112 and the VARD 130 for air entrapment. Unclamp and clamp the hemostat at the VARD purge line 141 as necessary to evacuate air.

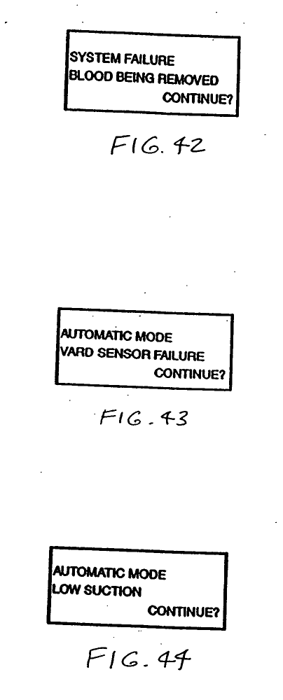

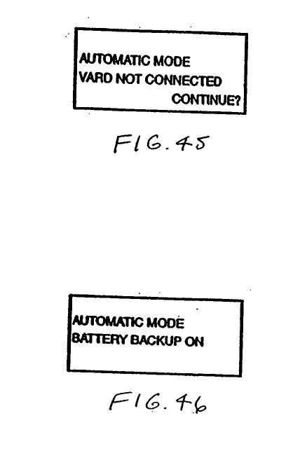

The user can perform the following procedure during a low suction state (FIG. 44): Confirm the wall vacuum regulator is set to −225 mmHg. Confirm the ¼ inch inner diameter suction lines between the regulator and the vacuum canister, and between the vacuum canister and the AAR controller 400 are connected, secure and functional. Confirm luer fittings and connections for the pressure line on the VARD air removal line 141 are secure. Confirm that the suction canister is not elevated. It should be on the floor. Confirm that the height of the pinch valve 410 on top of the AAR controller 400 is level with the VARD 130 height. Replace the air/separation filter on the vacuum monitoring line, if it is wetted out and no longer functional. Press RESET. If corrective action does not resolve the Alarm condition, call a local Technical Support representative and use a back-up AAR controller 400.

The user can perform the following procedure when the VARD 130 is not connected (FIG. 45): Check LEMO cable connections to the VARD and to the AAR controller 400. Confirm there is no fluid in the LEMO connections. If the message does not clear, replace cable. Press RESET. If corrective action do not resolve the Alarm condition, closely monitor the VARD 130 for the appearance for air. Press the MANUAL key or mechanical button 412 as necessary to remove air from the VARD 130. Call a local Technical Support representative and use a back-up AAR controller 400. FIG. 46 illustrates the screen when the battery back-up is on.

All patents and publications referenced herein are hereby incorporated by reference in their entireties. It will be understood that certain of the above-described structures, functions and operations of the above-described embodiments are not necessary to practice the invention and are included in the description simply for completeness of the described embodiments. It will also be understood that there may be other structures, functions and operations ancillary to the typical operation of mechanical instruments that are not disclosed and are not necessary to the practice of the invention. In addition, it will be understood that specifically described structures, functions and operations set forth in the above-referenced patents can be practiced in conjunction with the invention, but they are not essential to its practice. It is therefore to be understood, that within the scope of the appended claims, the invention may be practiced otherwise than as specifically described without actually departing from the spirit and scope of the invention.

1. A heart-lung machine for use with a patient, the heart-lung machine comprising:
a venous line receiving venous blood from the patient, the venous line being under negative pressure;
an arterial line supplying oxygenated blood to the patient;
a venous air removal device, a blood pump, a blood oxygenator and an arterial filter connected between the venous line and the arterial line, the blood pump being arranged to pump blood directly from said venous air removal device into the blood oxygenator; and
a disposable circuit support module for mounting the air removal device, the blood pump, and the blood oxygenator together and to a pole.

2. An air removal device for removing air from venous blood drawn from a patient, the air removal device comprising:
a blood intake;
an air-trapping device arranged to trap air present in the venous blood;
an inlet chamber positioned above the blood intake and arranged to receive air bubbles trapped by the air-trapping device; and
a sensor arranged to sense the presence of air in the inlet chamber;
a vacuum being applied to the inlet chamber when the sensor senses the presence of air in the inlet chamber so as to maintain the air removal device filled with blood but prevent blood from being aspirated into a purge line.

3. Apparatus for extracorporeal oxygenation of a patient's blood during cardiopulmonary bypass surgery, the apparatus comprising:
venous line means for receiving venous blood from a patient;
air removal means connected to the venous line means, for separating air from blood, the air removal means comprising an air chamber and means for diverting air entering the air removal means into the air chamber;
blood oxygenating means for oxygenating blood after it is drawn through the air filter means;
arterial line means for returning blood to the arterial system of the patient after the blood has been oxygenated by the blood oxygenating means;
an arterial blood filter in the arterial line means;
pump means for drawing blood through the venous line and air filter means and through the blood oxygenating means and arterial line means;
sensing means for sensing air in the air chamber; and
means for drawing air from the air chamber when air is sensed in the air chamber by the sensing means.

4. An extracorporeal blood circuit for use with a venous return line and an arterial line coupled to a patient, the extracorporeal blood circuit comprising:
a venous air removal device coupled to the venous return line, the venous air removal device performing an active air removal function;
a sensor that determines a blood level in the venous air removal device;
a purge line coupled to the venous air removal device;
a controller connected to the sensor, the controller causing the venous air removal device to perform the active air removal function through the purge line when the blood level is less than a threshold;
a pump coupled to the venous air removal device;
an oxygenator coupled to the pump; and
a blood filter coupled to the oxygenator and the arterial line.

5. The extracorporeal blood circuit of claim 4 wherein the pump and the oxygenator are disposable.

6. The extracorporeal blood circuit of claim 4 and further comprising a circuit support module that supports the venous air removal device, the pump, the oxygenator, the blood filter, and a plurality of fluid lines.

7. The extracorporeal blood circuit of claim 6 wherein the circuit support module is disposable.

8. The extracorporeal blood circuit of claim 6 wherein the circuit support module includes a C-shaped arm and a plurality of snap fittings.

9. The extracorporeal blood circuit of claim 6 and further comprising a system holder that can be coupled to a heart-lung machine and to the circuit support module.

10. The extracorporeal blood circuit of claim 9 wherein the system holder is reusable.

11. The extracorporeal blood circuit of claim 9 wherein the system holder includes a mast arm, an intravenous hanger coupled to the mast arm, a mast arm assembly that can be coupled to the heart-lung machine, an electronics arm assembly that can be coupled to the controller, and a support arm assembly that can be coupled to the circuit support module.

12. The extracorporeal blood circuit of claim 11 wherein the mast arm assembly can be moved along the mast arm.

13. The extracorporeal blood circuit of claim 4 and further comprising a pre-bypass loop that can connect the venous return line to the arterial line during at least one of priming and flushing.

14. The extracorporeal blood circuit of claim 4 and further comprising a measurement cell connected to the venous air removal device, the measurement cell measuring at least one of oxygen saturation and blood hematocrit.

15. The extracorporeal blood circuit of claim 4 and further comprising a venous blood pressure monitoring line, a pressure isolator, and a pressure monitor coupled to the venous return line.

16. The extracorporeal blood circuit of claim 4 and further comprising a venous filter purge line and a check valve coupled between the venous return line and a filter purge port of the blood filter.

17. The extracorporeal blood circuit of claim 4 and further comprising a passive vent coupled to the venous return line.

18. The extracorporeal blood circuit of claim 4 and further comprising a blood temperature monitoring adapter and a temperature probe coupled to the venous return line.

19. The extracorporeal blood circuit of claim 4 and further comprising a first Y-style line coupled to an outlet of the venous air removal device, an inlet of the pump, and a sequestering bag.

20. The extracorporeal blood circuit of claim 19 and further comprising a second Y-style line coupled to an outlet of the pump, an input of the oxygenator, and a prime solution holding bag.

21. The extracorporeal blood circuit of claim 4 wherein the pump is a centrifugal blood pump capable of providing negative pressure of up to approximately negative 200 mmHg.

22. The extracorporeal blood circuit of claim 4 wherein the pump includes a disposable portion and a reusable blood pump drive.

23. The extracorporeal blood circuit of claim 4 wherein the pump is located upstream of the venous air return device.

24. The extracorporeal blood circuit of claim 4 wherein the venous air removal device automatically removes air that collects in an upper part of the venous air removal device adjacent to a purge port connected to the purge line.

25. The extracorporeal blood circuit of claim 4 and further comprising a purge line segment coupled to the purge line, a fluid in-line sensor, a pinch valve, and the controller.

26. The extracorporeal blood circuit of claim 4 and further comprising a liquid trap coupled to the purge line in order to salvage red blood cells.

27. The extracorporeal blood circuit of claim 4 and further comprising a blood heat exchanger coupled to the oxygenator.

28. The extracorporeal blood circuit of claim 4 and further comprising an arterial blood sampling line coupled to an outlet of the oxygentor.

29. The extracorporeal blood circuit of claim 4 and further comprising a recirculation/cardioplegia line coupled to a Y-style line that is coupled to a recirculation port of the oxygenator, a sequestering bag, and one of a blood cardioplegia source and a hemoconcentrator.

30. The extracorporeal blood circuit of claim 4 wherein the blood filter removes air and microemboli.

31. The extracorporeal blood circuit of claim 4 wherein the blood filter includes a purge port coupled to the venous air removal device.

32. The extracorporeal blood circuit of claim 4 and further comprising a blood flow transducer coupled to the arterial line.

33. The extracorporeal blood circuit of claim 4 wherein substantially all of surfaces exposed to blood of the extracorporeal blood circuit are coated with a heparin coating.

34. The extracorporeal blood circuit of claim 4 wherein an operable flow rate through the extracorporeal blood circuit is up to approximately six liters per minute without producing substantial gas bubbles within at least one of the pump and the oxygenator.

35. A disposable circuit support module for use with an extracorporeal blood circuit including a venous air return device, a pump, an oxygenator, and a blood filter, the disposable circuit support module comprising:
a C-shaped arm; and
a plurality of snap fittings coupled to the C-shaped arm, each one of the plurality of snap fittings including a concave band rigidly coupled to the C-shaped arm and a movable U-shaped band that snaps into engagement with the concave band in order to engage one of the venous air return device, the oxygenator, and the blood filter.

36. The disposable circuit support module of claim 35 wherein at least one of the C-shaped arm and the plurality of snap fittings is substantially transparent.

37. The disposable circuit support module of claim 35 wherein the plurality of snap fittings includes a first lower snap fitting that supports the venous air return device, a second lower snap fitting that supports the oxygenator, and an upper snap fitting that supports the blood filter.

38. The disposable circuit support module of claim 35 and further comprising a plurality of raceways provided in the C-shaped arm into which fluid lines are press fit.

39. The disposable circuit support module of claim 37 wherein the second lower snap fitting is positioned so that an outlet of the pump is at approximately an equal level to an inlet of the oxygenator.

40. The disposable circuit support module of claim 37 wherein the first lower snap fitting is positioned so that the venous air removal device is above the pump; and the upper snap filter is positioned so that the blood filter is above the venous air removal device.

41. The disposable circuit support module of claim 35 wherein the pump can be independently manipulated with respect to the C-shaped arm.

42. A replacement assembly for use with an extracorporeal blood circuit, the replacement assembly comprising:
a circuit support module;
a venous air return device coupled to the disposable circuit support module;
a inlet/outlet pump manifold coupled to the venous air return device;
a oxygenator coupled to the pump and the disposable circuit support module; and
a blood filter coupled to the oxygenator and the disposable circuit support module.

43. The replacement assembly of claim 42 and further comprising a plurality of fluid lines and a plurality of quick connectors.

44. The replacement assembly of claim 42 wherein at least one of the circuit support module, the inlet/outlet pump manifold, and the oxygenator is disposable.

45. The replacement assembly of claim 42 wherein the circuit support module includes a plurality of snap fittings coupled to a C-shaped arm, each one of the plurality of snap fittings including a concave band rigidly coupled to the C-shaped arm and a movable U-shaped band that snaps into engagement with the concave band in order to engage one of the venous air return device, the oxygenator, and the blood filter.

46. The replacement assembly of claim 45 wherein at least one of the C-shaped arm and the plurality of snap fittings is substantially transparent.

47. The replacement assembly of claim 45 wherein the plurality of snap fittings includes a first lower snap fitting that supports the venous air return device, a second lower snap fitting that supports the oxygenator, and an upper snap fitting that supports the blood filter.

48. The replacement assembly of claim 45 and further comprising a plurality of raceways provided in the C-shaped arm into which fluid lines are press fit.

49. The replacement assembly of claim 45 wherein the second lower snap fitting is positioned so that an outlet of the pump is at approximately an equal level to an inlet of the oxygenator.

50. The replacement assembly of claim 45 wherein the first lower snap fitting is positioned so that the venous air removal device is above the pump; and the upper snap filter is positioned so that the blood filter is above the venous air removal device.

51. A method of priming an extracorporeal blood circuit, the method comprising:
connecting a venous return line to an arterial line using a pre-bypass loop;
preventing flow of prime solution into a venous air return device and a blood filter;
filling a pump and an oxygenator with prime solution in order to drive air bubbles upward and out of the pump and the oxygenator;
allowing prime solution to fill the venous return line and to pass into the venous return line after the pump and the oxygenator are filled with prime solution;
allowing prime solution to rise upward through the venous return line into the blood filter; and
coupling a vacuum source to a purge line coupled to the venous air removal device.

52. The method of claim 51 and further comprising visually inspecting the blood filter and the venous air removal device through transparent portions of the blood filter and the venous air removal device.

53. The method of claim 51 and further comprising venting air out of a blood filter purge line into the venous air return device; and suctioning air through a venous air return device purge line.

54. The method of claim 51 and further comprising disconnecting the pre-bypass loop and connecting an active air removal controller to the venous air return device.

55. A method of sensing and removing air and blood froth from an extracorporeal blood circuit including a venous air removal device, a pump, an oxygenator, and a blood filter, the method comprising:
connecting at least one piezoelectric crystal to the venous air removal device and to an active air removal controller;
sensing a level of blood in the venous air removal device; and
controlling the venous air removal device based on the level of blood in the venous air removal device in order to automatically remove air and blood froth when the level of blood falls below a threshold level.

56. The method of claim 55 and further comprising positioning four piezoelectric crystals in the venous air removal device, a first set of piezoelectric crystals being positioned above a second set of piezoelectric crystals, and using the second set of piezoelectric crystals if the first set of piezoelectric crystals fails.

57. The method of claim 55 and further comprising providing at least one of an audible alarm and a visual alarm when air and blood froth are being removed from the venous air removal device.
